(19) United States
(12) Patent Application Publication
(10) Pub. No.: US 2024/0391800 A1
(43) Pub. Date: Nov. 28, 2024

(54) HYPERSALINE DESALINATION USING IONIC LIQUIDS AND LOW-TEMPERATURE HEAT

(71) Applicant: UNIVERSITY OF NOTRE DAME DU LAC, South Bend, IN (US)

(72) Inventors: Tengfei Luo, South Bend, IN (US); Brandon Ashfeld, South Bend, IN (US); Jiaji Guo, South Bend, IN (US); Zachary Tucker, South Bend, IN (US); Eva Gulotty, South Bend, IN (US)

(21) Appl. No.: 18/693,020

(22) PCT Filed: Sep. 19, 2022
(86) PCT No.: PCT/US2022/043941

§ 371 Date: Mar. 18, 2024

Related U.S. Application Data
(60) Provisional application No. 63/246,277, filed on Sep. 20, 2021.

(51) Int. Cl. C02F 1/26; C02F 103/06; C02F 103/08; C07C 309/30; C07C 311/09; C07C 311/48; C07D 233/58; C07F 9/54

(52) U.S. Cl. CPC C02F 1/265; C07C 309/30; C07C 311/09; C07C 311/48; C07D 233/58; C07F 9/5407; C02F 2103/06; C02F 2103/08

(57) ABSTRACT

Described herein are compositions and methods for the low-temperature desalination of high-salinity water such as seawater or high-salt groundwater. In one embodiment, one or more of the ionic liquids, 1-ethyl-3-methylimidazolium bis(trifluoromethylsulfonyl)imide ([emim][Tf₂N]), 1-butyl-3-methylimidazolium bis(trifluoromethylsulfonyl) imide ([bmim][Tf₂N]), 1-ethyl-3-methylimidazolium tosy late ([emim][TsO]), tetrabutylphosphonium tosylate ([P₄₄₄₄][TsO]), tetrabutylphosphonium bis(trifluoromethylsulfonyl) imide ([P₄₄₄₄][Tf₂N]), trihexyltetradecylphosphonium bis(trifluoromethanesulfonyl) imide ([P₆₆₆₁₄][Tf₂N]), tributyloctylphosphonium bis(trifluoromethanesulfonyl) imide ([P₄₄₄₈][Tf₂N]). tributyloctylphosphonium benzo[d]imidazol-1-ide ([P₄₄₄₈][BzIm]), tributyloctylphosphonium trifluoromethanesulfonylleucine ([P₄₄₄₈][I-Leu]), 1-(methoxymethyl)-3-methyl-1H-imidazol-3-ium bis(trifluoromethylsulfonyl)imide ([mommim][Tf₂N]), or 1-eth-yl-3-methyl-1H-1,2,3-triazol-3-ium bis(trifluoromethylsulfonyl)imide ([1,2,3-emtriz][Tf₂N]), can be used to efficiently desalinate high salinity water.

### CROSS-REFERENCE TO RELATED APPLICATIONS

This application claims priority to U.S. Provisional Patent Application No. 63/246,277 filed on Sep. 20, 2021, which is incorporated by reference herein in its entirety.

### FEDERALLY SPONSORED RESEARCH

This invention was made with government support under grant numbers CBET 1510826, CBET 2031431, CHE 1665440, and CHE 1956170 awarded by the National Science Foundation. The government has certain rights in the invention.

### TECHNICAL FIELD

Described herein are compositions and methods for the low-temperature desalination of high-salinity water such as seawater or high-salt groundwater. In one embodiment, one or more of the ionic liquids, 1-ethyl-3-methylimidazolium bis(trifluoromethylsulfonyl)imide ([emim][Tf₂N]), 1-butyl-3-methylimidazolium bis(trifluoromethylsulfonyl)imide ([bmim][Tf₂N]), 1-ethyl-3-methylimidazolium tosylate ([emim][TsO]), tetrabutylphosphonium tosylate ([P₄₄₄₄][TsO]), tetrabutylphosphonium bis(trifluoromethylsulfonyl)imide ([P₄₄₄₄][Tf₂N]), trihexyltetradecylphosphonium bis(trifluoromethanesulfonyl)imide ([P₆₆₆₁₄][Tf₂N]), tributyloctylphosphonium bis(trifluoromethanesulfonyl)imide ([P₄₄₄₈][Tf₂N]), tributyloctylphosphonium benzo[d]imidazol-1-ide ([P₄₄₄₈][Bzim]), tributyloctylphosphonium trifluoromethanesulfonylleucine ([P₄₄₄₈][I-Leu]), 1-(methoxymethyl)-3-methyl-1H-imidazol-3-ium bis(trifluoromethylsulfonyl)imide ([mommim][Tf₂N]), or 1-ethyl-3-methyl-1H-1,2,3-triazol-3-ium bis(trifluoromethylsulfonyl)imide ([1,2,3-emtriz][Tf₂N]), can be used to efficiently desalinate high salinity water.

### BACKGROUND

The shortage of viable water resources is rapidly reaching critical status on a global scale. While extended droughts in many areas is a contributing factor, industrial and residential pollution of regional and local water supplies exacerbates this growing crisis. Given that ocean and subterranean saline aquifers contain 97.5% of the global water, desalination is a promising means for meeting freshwater demand. While membrane-based desalination processes like reverse osmosis (RO) have drawn considerable attention, the need for high grid electricity renders their application in low-resource settings challenging. RO can be energy-efficient (as low as 2 kWh/m³) in centralized plants largely due to the implementation of mechanical energy recovery systems, but at smaller scales, the energy cost can be much higher (up to 17 kWh/m³). In contrast, Directional Solvent Extraction (DSE) is an attractive alternative as it requires comparatively low operation temperatures relying almost exclusively on the consumption of waste heat or unconcentrated solar energy. The conceptual basis behind DSE centers on the use of a task-specific Directional Solvent (DS) that can solvate water in high yield, defined as the water solubility change per degree of temperature change (%/° C.), is insoluble in water, and will not solvate salt ions. It has been proposed that these directional solubilities are a result of the subtle balance between the hydrophilic and hydrophobic features of the solvent and the resulting intermolecular interactions with the solute. A major impediment to the implementation of DSE processes is the identification of an optimal directional solvent (DS). Currently, decanoic acid represents the best performing DS to date, with a water yield of only 0.027%/° C. This exceptionally low yield results in a low water production rate and relatively high energy consumption.

What is needed are compositions and methods for the low-temperature desalination of high-salinity water such as seawater or high-salt groundwater.

SUMMARY

One embodiment described herein is a method for desalinating water using a directional solvent extraction method (DSE), the method comprising: forming a mixture by combining a salt solution comprising one or more ionic salts and water with an ionic liquid, the ionic liquid comprising: an organic cation, wherein the organic cation is a phosphonium or an N-heterocyclic cation, and an anion, wherein the anion is a sulfonyl imide, a sulfonamide, a sulfonate, a carboxylate, an alkoxide, a hydroxide, a borate, a phosphate, a halide, or an N-heterocyclic anion; heating the mixture at a temperature of 40° C. to 90° C. to produce brine and an ionic solution, the ionic solution comprising water and the ionic liquid; removing the brine from the ionic solution; cooling the ionic solution to room temperature to produce a desalinated water phase and an ionic liquid phase: and removing the desalinated water phase. In one aspect, the ionic salt comprises Na⁺, K⁺, NH₄⁺, Ag⁺, Mg²⁺, Ca²⁺, Sr²⁺, Cd²⁺, Be²⁺, Ba²⁺, Pb²⁺, Hg²⁺, Zn²⁺, Cu²⁺, Ni²⁺, Cr³⁺, As³⁺, Tl³⁺, Al³⁺, Fe²⁺, Fe³⁺, Mn²⁺, Mn⁴⁺, Sb³⁺, Sb⁵⁺, or a combination thereof. In another aspect, the ionic salt comprises Cl⁻, Br⁻, I⁻, F⁻, NO₃⁻, NO₂⁻, CN⁻, PO₄³⁻, HPO₄²⁻, H₂PO₄⁻, PO₃³⁻, H₂PO₃⁻, H₂PO₃, SO₄²⁻, HSO₄⁻, CO₃²⁻, HCO₃⁻, SiO₃²⁻, HSiO₃⁻, HBO₃²⁻, or H₂BO₃⁻, or a combination thereof. In another aspect, the ionic salt comprises NaCl, KCl, MgCl₂, CaCl₂, NaH₂PO₄, Na₂HPO₄, Na₃PO₄, NaHSO₄, Na₂SO₄, NaH₂PO₄, Na₂HPO₄, Na₃PO₄, NaHSO₄, Na₂SO₄, or a combination thereof. In another aspect, the salt solution comprises 3-15% by mass (wt %) of the ionic salt. In another aspect, heating the mixture occurs at a temperature of 45° C. to 75° C. In another aspect, the method further comprises recycling the ionic liquid phase. In another aspect, the organic cation is a phosphonium cation. In another aspect, the phosphonium cation is a tetraalkylphosphonium cation. In another aspect, the tetraalkylphosphonium cation is a tetralkyl phosphonium of formula:

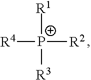

wherein R¹, R², R³, and R⁴ are each independently C₁₋₂₀alkyl. In another aspect, R¹, R², and R³ are each independently C₄ salkyl and R⁴ is C₄₋₁₄alkyl. In another aspect, R¹, R², R³, and R⁴ are each C₄alkyl. In another aspect, R¹, R², and R³ are each C₄alkyl and R⁴ is C₈alkyl. In another aspect, R¹, R², and R³ are each Csalkyl and R⁴ is C₁₄alkyl. In another aspect, the organic cation is an N-heterocyclic cation. In another aspect, the N-heterocyclic cation is an imidazolium cation of formula:

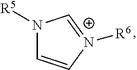

wherein R⁵ is C₁₄alkyl and Re is C₁₋₁₀alkyl or -C₁₋₃alkylene-OC₁₋₄alkyl. In another aspect, R⁵ is methyl and R⁶ is C₁₋₅alkyl or —CH₂-OC₁₋₄alkyl. In another aspect, R⁶ is C₂₋₄alkyl or —CH₂-OC₁₋₂alkyl. In another aspect, the N-heterocyclic cation is a triazolium cation of formula:

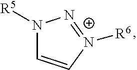

wherein R⁵ is C₁₋₄alkyl and R⁶ is C₁₋₁₀alkyl or -C₁₋₃alkylene-OC₁₋₄alkyl. In another aspect, R⁵ is methyl and R⁶ is C₂₋₄alkyl. In another aspect, the anion is bis(trifluoromethylsulfonyl)imide ([Tf₂N]⁻) or tosylate ([OTs]⁻). In another aspect, the anion is [Tf₂N]⁻. In another aspect, the anion is [OTs]⁻. In another aspect, the ionic liquid is:

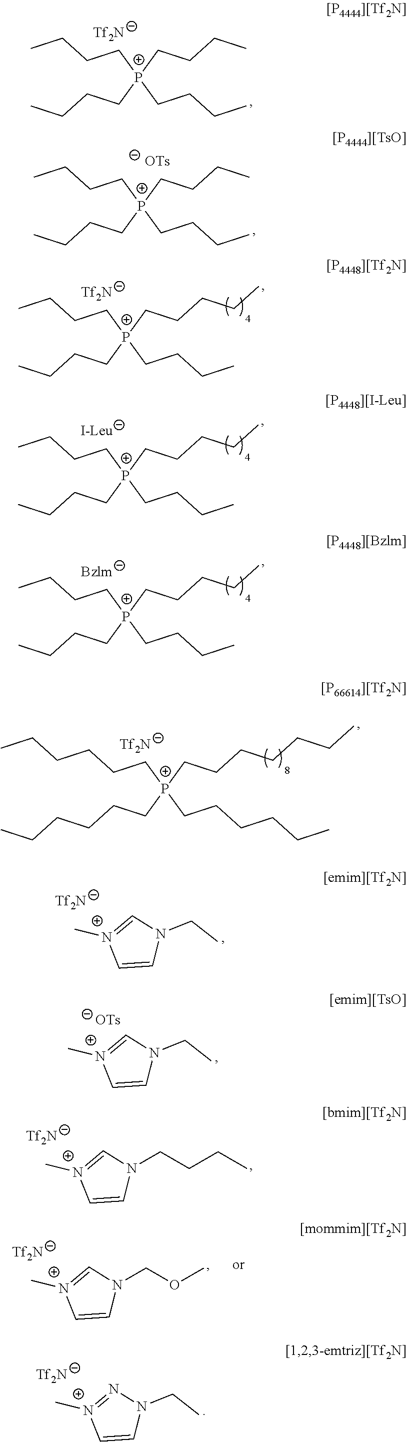

In another aspect, the ionic liquid has an ion rejection rate of at least 70%.
In another aspect, the ionic liquid has an ion rejection rate of at least 80%.
In another aspect, the ionic liquid has an ion rejection rate of at least 90%.

Another embodiment described herein is freshwater produced by any of the methods described herein.

Another embodiment described herein is the use any of the methods described herein to produce freshwater.

Another embodiment described herein is a method for desalinating seawater or groundwater with dissolved ionic salts, the method comprising: forming a mixture by combining seawater or groundwater comprising dissolved ionic salts with an ionic liquid, wherein the ionic liquid is:

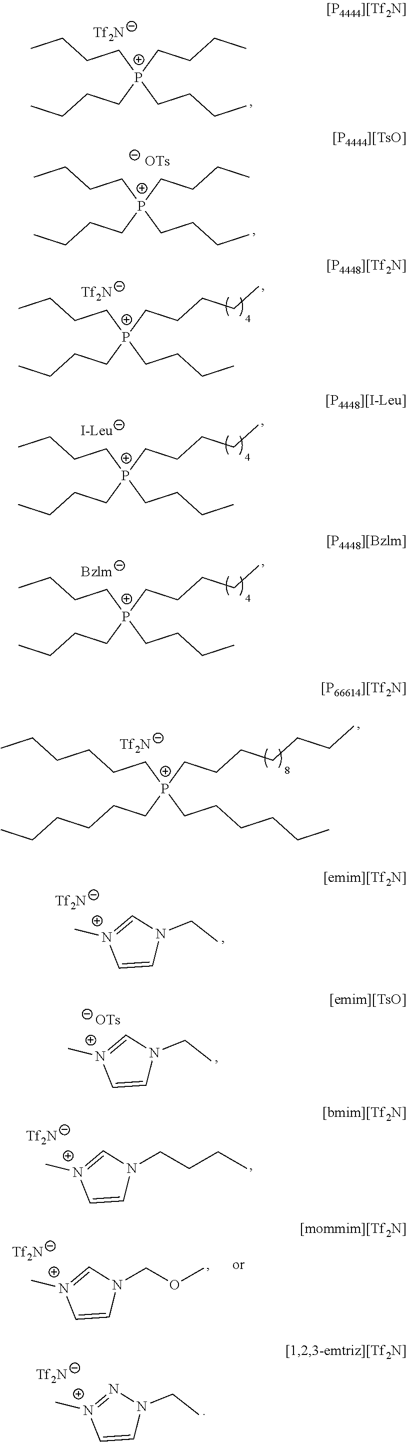

heating the mixture at a temperature of 40° C. to 90° C. to produce brine and an ionic solution, the ionic solution comprising water and the ionic liquid; removing the brine from the ionic solution; cooling the ionic solution to room temperature to produce a desalinated water phase and an ionic liquid phase: and removing the desalinated water phase.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows Directional Solvent Extraction (DSE) desalination employing Task-Specific lonic Liquids (TSILs). An optimal Directional Solvent lonic Liquid (DS IL) would display sparing solubility with highly concentrated salt water (a), which then upon heating, draws only water into the IL while leaving the salts in the water phase yielding a high salinity brine (MX) (b). Removal of the MX byproduct (c) followed by a reduction in temperature leads to decreased solubility of water in the IL (d) that could then be readily separated to provide the desired fresh water. Upon separation, the recovered IL is recycled for subsequent process turnover of the DSE cycle (e). FIG. 1B shows the experimental DSE procedure of water desalination using ionic liquids.

FIG. 2 shows the Task-Specific lonic Liquids (TSILs) for Directional Solvent Extraction (DSE) evaluation.

FIGS. 3A-C show water yield and salt rejection observed. FIG. 3A shows the water yield and temperature relationship of [1-ethyl-3-methyl-1H-imidazol-3-imidazol-3-ium][bis(trifluorosulfonyl)imide] ([emim][Tf₂N]) and decanoic acid. Error bar is the standard deviation of different tests for each condition. FIG. 3B shows the desalination of moderate salt content water (3.7-5.0 wt %). FIG. 3C shows the desalination of high salt content water (10.8 wt %). Each bar in FIG. 3B and FIG. 3C corresponds to one experiment.

FIGS. 4A-C show a Molecular Dynamics (MD) simulation of the interface between IL and NaCl water solution (3.7%). FIG. 4A shows snapshots of MD simulations at different times, where the blue block is water, the red block is IL, and the large pink and dark blue spheres are Cl⁻and Na+ions, respectively. FIG. 4B shows density profiles of water and [Tf₂N]⁻ of the IL at different times corresponding to the snapshots in FIG. 4A. The [emim]⁺ shows similar profile as the [Tf₂N]⁻ and it is not plotted for clarity of the figure. FIG. 4C shows the number of hydrogen bonds (H-bonds) between [Tf₂N]⁻ and water molecules as a function of time.

FIG. 5 shows a comparison of exergy consumption of directional solvent extraction (DSE) using [emim][Tf₂N] and decanoic acid and multi-stage flash (MSF). Propagated from the data uncertainty from FIG. 3, the calculated uncertainty of the blue data points ranges from 20% (at 45° C.) to 17% (75° C.).

FIG. 6 hows schematics of a sodium ion “gradually appearing” in a solvent for calculating the free energy difference using MD simulations and thermodynamic integration. Here, λ is a coupling factor that tunes the interatomic interactions between the ion and solvent, with λ=1 corresponding to the non-interactions state and 80 =0 corresponding to the fully interacting states.

FIG. 7 shows structures of ionic liquids. FIG. 7A shows 1-ethyl-3-methyl-1H-imidazol-3-imidazol-3-ium ([emim]⁺). FIG. 7B shows 1-(methoxymethyl)-3-methyl-1H-imidazol-3-ium ([mommim]⁺). FIG. 7C shows 1-ethy-3-methyl-1H-1,2,3-triazol-3-ium. FIG. 7D shows bis(trifluoromethylsulfonyl)imide ([Tf₂N]⁻).

FIGS. 8A-B shows the calculated solvation free energies of NaCl in selected ionic liquids employing TIP4P. FIG. 8A shows the calculated solvation free energy of NaCl in [mom-mim][Tf₂N]. FIG. 8B shows the calculated solvation free energy of NaCl in [1,2,3-emtriz][Tf₂N].

FIGS. 9A-C show MD simulation of the interface between [mommim][Tf₂N] and 25.8% NaCl water solution. FIG. 9A shows snapshots of MD simulation at 0 ns, 5 ns and 30 ns, respectively, where the cyan block is water, the red and pink are [mommim]⁺ and [Tf₂N]⁻ respectively, and the violet and orange spheres are Na⁺ and Cl⁻, respectively. FIG. 9B shows density distribution of water and [Tf₂N]⁻ of the IL at 0 ns, 5 ns, and 30 ns. FIG. 9C shows water and number of hydrogen bonds formed between [Tf₂N]⁻ and water relationship as a function of time.

FIGS. 10A-F show MD simulation of the interface between [mommim][Tf₂N] and [1,2,3-emtriz][Tf₂N] with 3.7% NaCl water solution. FIG. 10A shows [mommim][Tf₂N], snapshots of MD simulation at 0 ns, 5 ns and 30 ns, respectively, where the cyan block is water, red and pink are [mommim]⁺ and [Tf₂N]⁻ respectively, and the violet and orange spheres are Na⁺ and Cl⁻, respectively. FIG. 10B shows [1,2,3-emtriz][Tf₂N], Snapshots of MD simulation at 0 ns, 5 ns and 30 ns, respectively, where the cyan block is water, red and pink part are 1-ethyl-3-methyl-1H-1,2,3-triazol-3-ium]⁺ and [Tf₂N]⁻ respectively, and the violet and orange spheres are Na⁺ and Cl⁻, respectively. FIG. 10C shows [mommim][Tf₂N], density distribution of water and [Tf₂N]⁻ of the IL at 0 ns, 5 ns, and 30 ns. FIG. 10D shows [1,2,3-emtriz][Tf₂N], density distribution of water and [Tf₂N]⁻ of the IL at 0 ns, 5 ns, and 30 ns. FIG. 10E shows [mommim][Tf₂N]. water and number of hydrogen bonds formed between [Tf₂N]⁻ and water relationship as a function of time. FIG. 10F shows [1,2,3-emtriz][Tf₂N], water and number of hydrogen bonds formed between [Tf₂N]⁻ and water relationship as a function of time.

FIG. 11 shows a comparison of exergy consumption of DSE using [mommim][Tf₂N] and [1,2,3-emtriz][Tf₂N] and MSF.

### DETAILED DESCRIPTION

Unless otherwise defined, all technical and scientific terms used herein have the same meaning as commonly understood by one of ordinary skill in the art. For example, any nomenclatures used in connection with, and techniques of chemistry and biochemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold. of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “freshwater” is water containing less than 1,000 milligrams per liter (mg/L) of solids (e.g., ionic salts).

lonic liquids (ILs) have demonstrated exceptional promise as a molecular framework for the development of task-specific fluids due to their design flexibility and inherently low volatility. ILs comprised of organic ions residing in a liquid state between room temperature and 100° C. have shown promise as working fluids across a variety of applications, including the separation of organic compounds, sequestration of transition metals, capturing carbon dioxide, and desalinating aqueous media. The structural versatility and variability of many ILs enable extraordinary freedom in solvent design, as the cation and anion components can be individually engineered to achieve the desired directional solubilities.

Definitions of specific functional groups and chemical terms are described in more detail below. For purposes of this disclosure, the chemical elements are identified in accordance with the Periodic Table of the Elements. CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkoxyfluoroalkyl,” as used herein, refers to an alkoxy group, as defined herein, appended to the parent molecular moiety through a fluoroalkyl group, as defined herein.

The term “alkylene,” as used herein, refers to a divalent group derived from a straight or branched chain hydrocarbon of 1 to 10 carbon atoms, for example, of 2 to 5 carbon atoms. Representative examples of alkylene include, but are not limited to, —CH₂—, —CD₂—, —CH₂CH₂—, —CH₂CH₂CH₂—, —CH₂CH₂CH₂CH₂—, and-CH₂CH₂CH₂CH₂CH₂—.

The term “alkylamino,” as used herein, means at least one alkyl group, as defined herein, is appended to the parent molecular moiety through an amino group, as defined herein.

The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo [d][1,3] dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9-to 12-membered fused bicyclic system).

The term “cyanoalkyl,” as used herein, means at least one —CN group, is appended to the parent molecular moiety through an alkylene group, as defined herein.

The term “cyanofluoroalkyl,” as used herein, means at least one —CN group, is appended to the parent molecular moiety through a fluoroalkyl group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl).

Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl).

Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms cycloalkylene and heterocyclylene refer to divalent groups derived from the base ring, i.e., cycloalkane, heterocycle. For purposes of illustration, examples of cycloalkylene and heterocyclylene include, respectively,

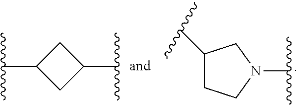 [SMILES: *C1CC(*)C1 ; *C1CCN(*)C1]

Cycloalkylene and heterocyclylene include a geminal divalent groups such as 1,1-C₃₋₆cycloalkylene (i.e.,

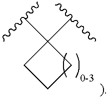

A further example is 1,1-cyclopropylene (i.e.,

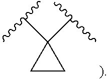

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “fluoroalkylene,” as used herein, means an alkylene group, as defined herein, in which one, two, three, four, five, six, seven or eight hydrogen atoms are replaced by fluorine. Representative examples of fluoroalkyl include, but are not limited to —CF₂—, —CH₂CF₂—, 1,2-difluoroethylene, 1,1.2,2-tetrafluoroethylene. 1,3,3,3-tetrafluoropropylene, 1,1,2,3,3-pentafluoropropylene, and perfluoropropylene such as 1,1,2,2,3,3-hexafluoropropylene.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides. The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g., 1, 2, 3, or 4 heteroatoms independently selected from O, S, and N). The five membered aromatic monocyclic rings have two double bonds, and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3.2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl,

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three-or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven-and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro [3.3]heptan-2-yl, 2-oxa-6-azaspiro [3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta [c] pyrrolyl, octahydropyrrolopyridinyl, tetrahydroisoquinolinyl, 7-oxabicyclo [2.2.1]heptanyl, hexahydro-2H-cyclopenta[b]furanyl, 2-oxaspiro [3.3]heptanyl, 3-oxaspiro [5.5]undecanyl, 6-oxaspiro [2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta [b]furan, hexahydro-1H-1,4-methanocyclopenta[c]furan, aza-adamantane (1-azatricyclo [3.3.1.13,7]decane), and oxa-adamantane (2-oxatricyclo [3.3.1.13.7]decane). The monocyclic, bicyclic, and tricyclic heterocyclyls are connected to the parent molecular moiety at a non-aromatic ring atom.

The term “hydroxyl” or “hydroxy.” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C_{1-4,”} the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

The term “cation,” as used herein, means an ionic species with a positive charge.

The term “anion,” as used herein, means an ionic species with a negative charge.

The term “N-heterocyclic cation,” as used herein, means a heterocycle containing at least one nitrogen (N) atom, wherein the N-containing heterocycle has a positive charge.

The term “N-heterocyclic anion,” as used herein, means a heterocycle containing at least one nitrogen (N) atom, wherein the N-containing heterocycle has a negative charge.

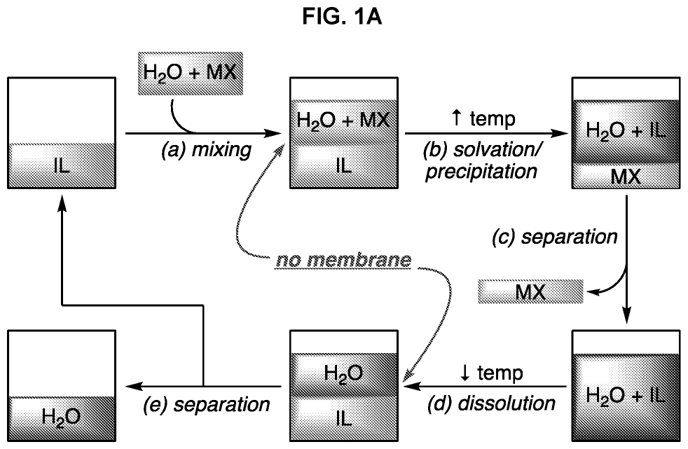

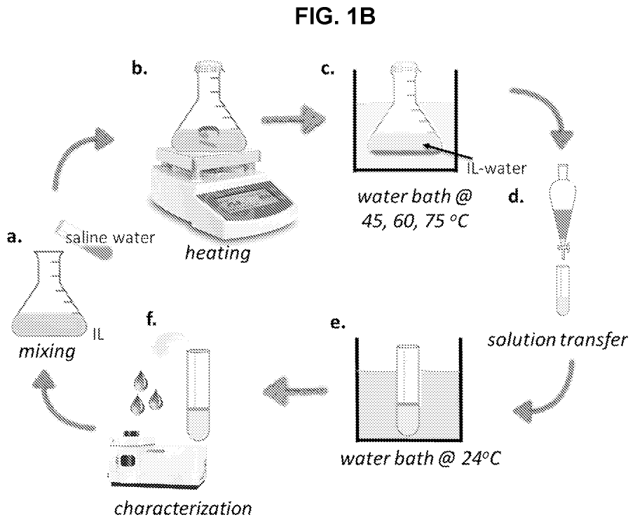

As illustrated in FIG. 1, an optimal Directional Solvent (DS) lonic liquid (IL) would display sparing solubility with highly concentrated salt water (a), which then upon heating, draws only water into the IL while leaving the salts in the water phase yielding a high salinity brine (MX) (b). Removal of the MX byproduct (c) followed by a reduction in temperature leads to decreased solubility of water in the IL (d) that could then be readily separated to provide the desired fresh water. Upon separation, the recovered IL is recycled for subsequent process turnover of the DSE cycle (e). Given the variable water and salt solubility of many ILs, a challenge was to identify a suitable ionic liquid that would accommodate each phase of this DSE cycle.

One embodiment described herein is a method for desalinating water using a directional solvent extraction method (DSE), the method comprising: forming a mixture by combining a salt solution comprising one or more ionic salts and water with an ionic liquid, the ionic liquid comprising: an organic cation, wherein the organic cation is a phosphonium or an N-heterocyclic cation, and an anion, wherein the anion is a sulfonyl imide, a sulfonamide, a sulfonate, a carboxylate, an alkoxide, a hydroxide, a borate, a phosphate, a halide, or an N-heterocyclic anion; heating the mixture at a temperature of 40° C. to 90° C. to produce brine and an ionic solution, the ionic solution comprising water and the ionic liquid; removing the brine from the ionic solution; cooling the ionic solution to room temperature to produce a desalinated water phase and an ionic liquid phase: and removing the desalinated water phase. In one aspect, the ionic salt comprises Na⁺, K⁺, NH⁴⁺, Ag⁺, Mg²⁺, Ca²⁺, Sr²⁺, Cd²⁺, Be²⁺, Ba²⁺, Pb²⁺, Hg²⁺, Zn²⁺, Cu²⁺, Ni²⁺, Cr³⁺, As³⁺, Tl³⁺, Al³⁺, Fe²⁺, Fe³⁺, Mn²⁺, Mn⁴⁺, Sb³⁺, Sb⁵⁺, or a combination thereof. In another aspect, the ionic salt comprises Cl⁻, Br⁻, I⁻, , F⁻, NO₃⁻, NO₂⁻, CN⁻, PO₄³⁻, HPO₄²⁻, H₂PO₄⁻, PO₃³⁻, HPO₃²⁻, H₂PO₃, SO₄²⁻, HSO₄⁻, CO₃²⁻, HCO₃⁻, SiO₃²⁻, HSiO₃⁻, HBO₃²⁻, or H₂BO₃⁻, or a combination thereof. In another aspect, the ionic salt comprises NaCl, KCl, MgCl₂, CaCl₂, NaH₂PO₄, Na₂HPO₄, Na₃PO₄, NaHSO₄, Na₂SO₄, NaH₂PO₄, Na₂HPO₄, Na₃PO₄, NaHSO₄, Na₂SO₄, or a combination thereof. In another aspect, the salt solution comprises 3-15% by mass (wt %) of the ionic salt. In another aspect, heating the mixture occurs at a temperature of 45° C. to 75° C. In another aspect, the method further comprises recycling the ionic liquid phase. In another aspect, the organic cation is a phosphonium cation. In another aspect, the phosphonium cation is a tetraalkylphosphonium cation. In another aspect, the tetraalkylphosphonium cation is a tetralkyl phosphonium of formula:

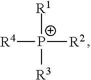

wherein R¹, R², R³, and R⁴ are each independently C₁₋₂₀alkyl. In another aspect, R¹, R², and R³ are each independently C₄ alkyl and R⁴ is C₄₋₁₄alkyl. In another aspect, R¹, R², R³, and R⁴ are each C₄alkyl. In another aspect, R¹, R², and R³ are each C₄alkyl and R⁴ is C₈alkyl. In another aspect, R¹, R², and R³ are each Csalkyl and R⁴ is C₁₄alkyl. In another aspect, the organic cation is an N-heterocyclic cation. In another aspect, the N-heterocyclic cation is an imidazolium cation of formula:

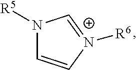

wherein R⁵ is C₁₋₄alkyl and R⁶ is C₁₋₁₀alkyl or —C₁₋₃alkylene-OC₁₋₄alkyl. In another aspect, R⁵ is methyl and R⁶ is C₁₋₅alkyl or —CH₂-OC₁₋₄alkyl. In another aspect, R⁶ is C₂₋₄alkyl or —CH₂-OC₁₋₂alkyl. In another aspect, the N-heterocyclic cation is a triazolium cation of formula:

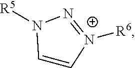

wherein R⁵ is C₁₋₄alkyl and R⁶ is C₁₋₁₀alkyl or —C₁₋₃alkylene-OC₁-4alkyl. In another aspect, R⁵ is methyl and R⁶ is C₂₋₄alkyl. In another aspect, the anion is bis(trifluoromethylsulfonyl)imide ([Tf₂N]⁻) or tosylate ([OTs]⁻). In another aspect, the anion is [Tf₂N]⁻. In another aspect, the anion is [OTs]⁻. In another aspect, the ionic liquid is:

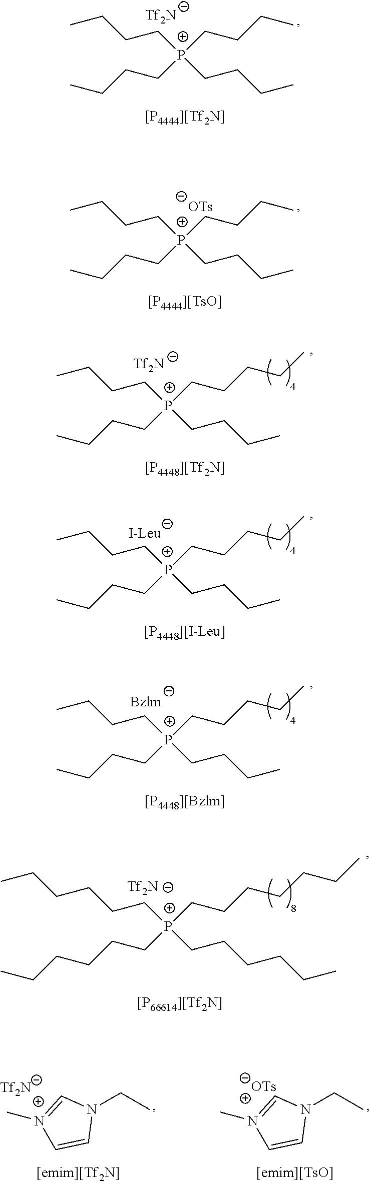

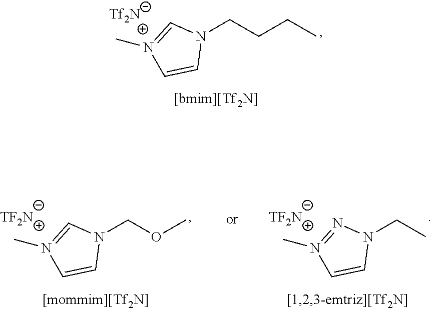

In another aspect, the ionic liquid has an ion rejection rate of at least 70%.
In another aspect, the ionic liquid has an ion rejection rate of at least 80%.
In another aspect, the ionic liquid has an ion rejection rate of at least 90%.

Another embodiment described herein is freshwater produced by any of the methods described herein.

Another embodiment described herein is the use any of the methods described herein to produce freshwater.

Another embodiment described herein is a method for desalinating seawater or groundwater with dissolved ionic salts, the method comprising: forming a mixture by combining seawater or groundwater comprising dissolved ionic salts with an ionic liquid, wherein the ionic liquid is:

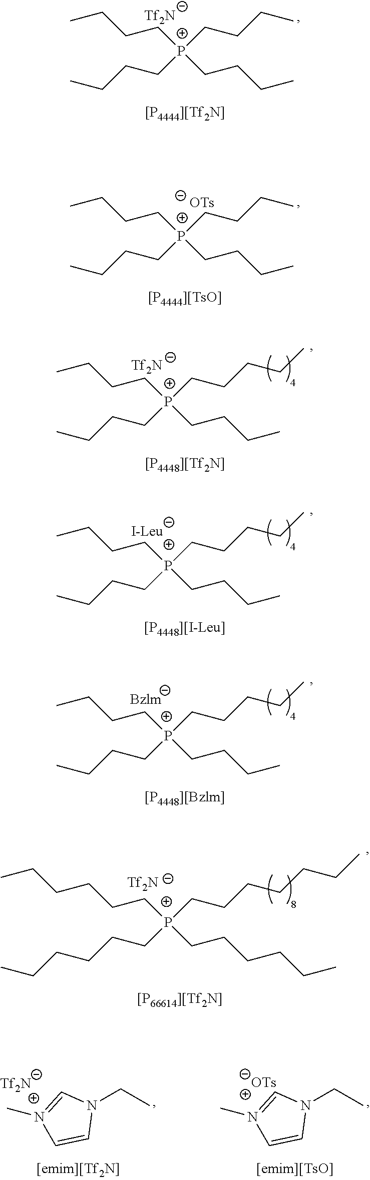

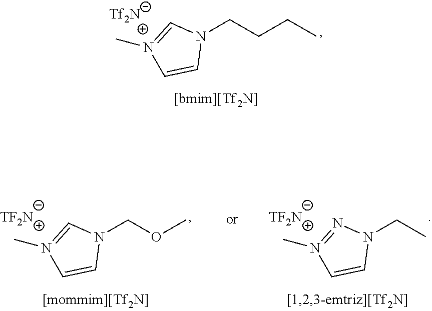

heating the mixture at a temperature of 40° C. to 90° C. to produce brine and an ionic solution, the ionic solution comprising water and the ionic liquid; removing the brine from the ionic solution; cooling the ionic solution to room temperature to produce a desalinated water phase and an ionic liquid phase: and removing the desalinated water phase.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for desalinating water using a directional solvent extraction method (DSE), the method comprising:
   forming a mixture by combining a salt solution comprising one or more ionic salts and water with an ionic liquid, the ionic liquid comprising:
      an organic cation, wherein the organic cation is a phosphonium or an N-heterocyclic cation, and
      an anion, wherein the anion is a sulfonyl imide, a sulfonamide, a sulfonate,
         a carboxylate, an alkoxide, a hydroxide, a borate, a phosphate, a halide, or an N-heterocyclic anion;
heating the mixture at a temperature of 40° C. to 90° C. to produce brine and an ionic solution, the ionic solution comprising water and the ionic liquid;
removing the brine from the ionic solution;
cooling the ionic solution to room temperature to produce a desalinated water phase and an ionic liquid phase; and removing the desalinated water phase.

Clause 2. The method of clause 1, wherein the ionic salt comprises Nat, K⁺, NH⁴⁺, Ag⁺, Mg²⁺, Ca²⁺, Sr²⁺, Cd²⁺, Be²⁺, Ba²⁺, Pb²⁺, Hg²⁺, Zn²⁺, Cu²⁺, Ni²⁺, Cr³⁺, As³⁺, Tl³⁺, Al³⁺, Fe²⁺, Fe³⁺, Mn²⁺, Mn⁴⁺, Sb³⁺, Sb⁵⁺, or a combination thereof.

Clause 3. The method of clause 1 or 2, wherein the ionic salt comprises Cl⁻, Br⁻, I⁻, F⁻, NO₃⁻, NO₂⁻, CN⁻, PO₄³⁻, HPO₄²⁻, H₂PO₄⁻, PO₃³⁻, HPO₃²⁻, H₂PO₃₋, SO₄²⁻, HSO₄⁻, CO₃²⁻, HCO₃⁻, SiO₃²⁻, HSiO₃⁻, HBO₃²⁻, or H₂BO₃⁻, or a combination thereof.

Clause 4. The method of any one of clauses 1-3, wherein the ionic salt comprises NaCl, KCl, MgCl₂, CaCl₂, NaH₂PO₄, Na₂HPO₄, Na₃PO₄, NaHSO₄, Na₂SO₄, NaH₂PO₄, Na₂HPO₄, Na₃PO₄, NaHSO₄, Na₂SO₄, or a combination thereof.

Clause 5. The method of any one of clauses 1-4, wherein the salt solution comprises 3-15% by mass (wt %) of the ionic salt.

Clause 6. The method of any one of clauses 1-5, wherein heating the mixture occurs at a temperature of 45° C. to 75° C.

Clause 7. The method of any one of clauses 1-6, wherein the method further comprises recycling the ionic liquid phase.

Clause 8. The method of any one of clauses 1-7, wherein the organic cation is a phosphonium cation.

Clause 9. The method of any one of clauses 1-8, wherein the phosphonium cation is a tetraalkylphosphonium cation.

Clause 10. The method of any one of clauses 1-9, wherein the tetraalkylphosphonium cation is a tetralkyl phosphonium of formula:

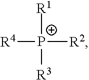

wherein R¹, R², R³, and R⁴ are each independently C₁₋₂₀alkyl.

Clause 11. The method of any one of clauses 1-10, wherein, R¹, R², and R³ are each independently C₄₋₆alkyl and R⁴ is C₄₋₁₄alkyl.

Clause 12. The method of any one of clauses 1-11, wherein R¹, R², R³, and R⁴ are each C₄alkyl.

Clause 13. The method of any one of clauses 1-12, wherein R¹, R², and R³ are each C₄alkyl and R⁴ is Calkyl.

Clause 14. The method of any one of clauses 1-13, wherein R¹, R², and R³ are each Cealkyl and R⁴ is C₁₄alkyl.

Clause 15. The method of any one of clauses 1-14, wherein the organic cation is an N-heterocyclic cation.

Clause 16. The method of any one of clauses 1-15, wherein the N-heterocyclic cation is an imidazolium cation of formula:

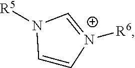

wherein R⁵ is C₁₋₄alkyl and R⁶ is C₁₋₁₀alkyl or —C₁₋₃alkylene-OC₁₋₄alkyl.

Clause 17. The method of any one of clauses 1-16, wherein R⁵ is methyl and R⁶ is C₁₋₅alkyl or —CH₂—OC₁₋₄alkyl.

Clause 18. The method of any one of clauses 1-17, wherein R⁶ is C₂₋₄alkyl or —CH₂—OC₁₋₂alkyl.

Clause 19. The method of any one of clauses 1-18, wherein the N-heterocyclic cation is a triazolium cation of formula:

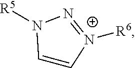

wherein R⁵ is C₁₋₄alkyl and R⁶ is C₁₋₁₀alkyl or —C₁₋₃alkylene-OC₁₋₄alkyl.

Clause 20. The method of any one of clauses 1-19, wherein R⁵ is methyl and R⁶ is C₂₋₄alkyl.

Clause 21. The method of any one of clauses 1-20, wherein the anion is bis(trifluoromethylsulfonyl)imide ([Tf₂N]⁻) or tosylate ([OTs]⁻).

Clause 22. The method of any one of clauses 1-21, wherein the anion is [Tf₂N]⁻.

Clause 23. The method of any one of clauses 1-21, wherein the anion is [OTs]⁻.

Clause 24. The method of any one of clauses 1-23, wherein the ionic liquid is:

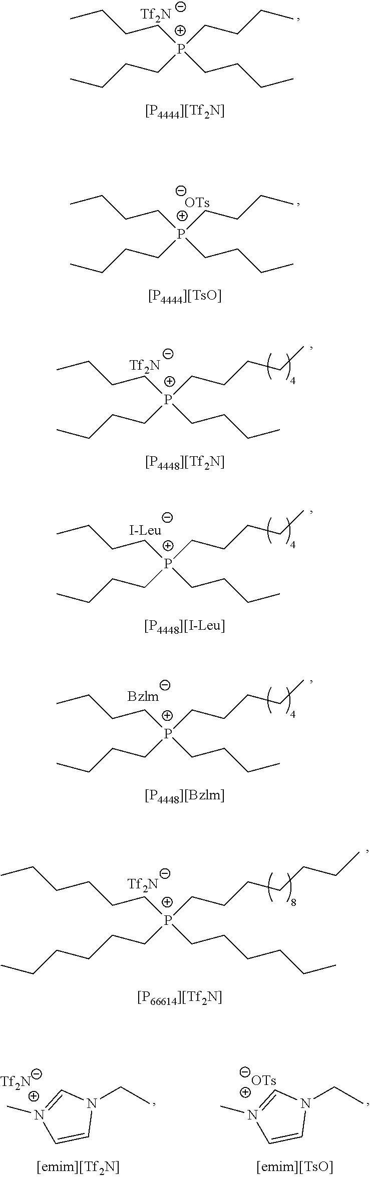

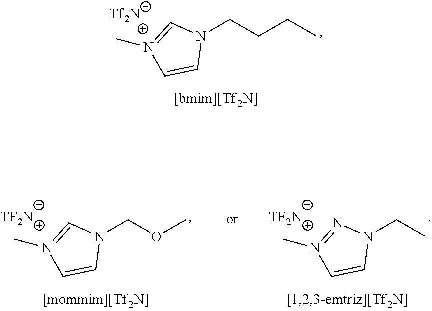

Clause 25. The method of any one of clauses 1-24, wherein the ionic liquid has an ion rejection rate of at least 70%.

Clause 26. The method of any one of clauses 1-25, wherein the ionic liquid has an ion rejection rate of at least 80%.

Clause 27. The method of any one of clauses 1-26, wherein the ionic liquid has an ion rejection rate of at least 90%.

Clause 28. Freshwater produced by the method of any one of clauses 1-27.

Clause 29. Use of the method of any one of clauses 1-27 to produce freshwater.

Clause 30. A method for desalinating seawater or groundwater with dissolved ionic salts, the method comprising:

forming a mixture by combining seawater or groundwater comprising dissolved ionic salts with an ionic liquid, wherein the ionic liquid is:

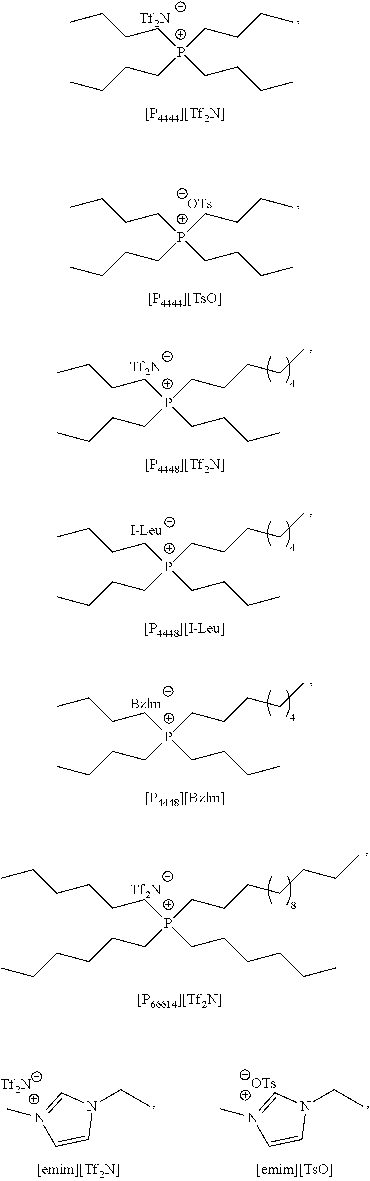

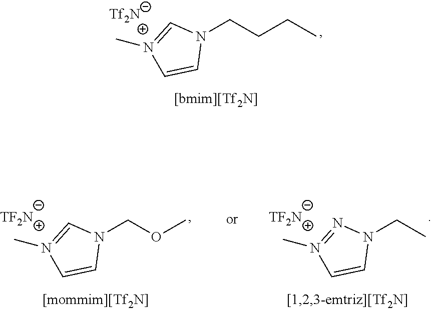

heating the mixture at a temperature of 40° C. to 90° C. to produce brine and an ionic solution, the ionic solution comprising water and the ionic liquid; removing the brine from the ionic solution; cooling the ionic solution to room temperature to produce a desalinated water phase and an ionic liquid phase; and removing the desalinated water phase.

### EXAMPLES

### Example 1

Synthesis and Characterization of lonic Liquids

All organic solvents were distilled under and argon atmosphere and passed through a column of molecular sieves prior to use. Deionized water was used for all reactions unless otherwise stated. Reagents were used as received by commercial sources without further purification. All reactions were carried out in oven dried glassware under nitrogen or argon at room temperature unless otherwise specified. Compounds were characterized by ¹H nuclear magnetic resonance (NMR) spectra obtained at 400 or 500 MHz, 13C NMR obtained at 100 or 125 MHZ, and ¹⁹F NMR obtained at 376 MHz. Chemical shifts are reported in parts per million (ppm, δ), and referenced from the solvent. Coupling constants are reported in Hertz (Hz). Spectral splitting patterns are designated as s, singlet; d, doublet; t, triplet; q, quartet; m, multiplet; comp, complex; app, apparent; and br, broad. Infrared (IR) spectra were obtained using a Thermo Electron Nicolet 380 FT-IR using a silicon (Si) crystal in an attenuated total reflectance (ATR) tower and reported as wavenumbers (cm⁻¹). High-and Low-resolution electrospray ionization (ESI) measurements were made with a JEOL JMS-AX505HA mass spectrometer. Ionic Liquids: [emim][Tf₂N], [P₄₄₄₄][Tf₂N], [P₄₄₄₄][TsO], [P₄₄₄₈][Benzlm] and compounds tributyloctyl phosphonium bromide, 1-butyl-3-methylimidazolium iodide, and N-trifluoromethanesulfonylleucine methyl ester (I-Leu) were synthesized by reported methods. See e.g., Srour et al., Green Chem. 15:1341-1347 (2013); Weber et al., Org. Biomol. Chem. 11:2534-2542 (2013); Kamio et al., J. Membr. Sci. 570:93-102 (2019); Tsuji & Ohno, Chem. Lett. 42:527-529 (2013); Rauber et al., Phys.

Chem. Chem. Phys. 19:27251-27258 (2017); Tröger-Müller et al., Phys. Chem. Chem. Phys. 20:11437-11443 (2018); Fukumoto & Ohno, Chem. Comm., 3081-3083 (2006), respectively.

General Synthesis of Tetraalkylphosphonium bis(trifluoromethylsulfonyl)imide Ionic Liquids

To a 100 mL round bottom flask equipped with a magnetic stir bar was added tetraalkylphosphonium bromide (10 mmol, 1 equiv.), placed under an atmosphere of nitrogen, and diluted with H₂O (40 mL). To the solution of tetraalkylphosphonium bromide was added LiTf₂N (10 mmol, 1 equiv.) as a 1 M solution in H₂O. The mixture was stirred at rt for 15 hours then extracted with CH₂Cl₂ (3×20 mL). The combined organic fractions were washed with H₂O (3×10 mL), dried, (MgSO₄), filtered, and concentrated under reduced pressure. The ionic liquid was then dried further for an additional 12 hours in a vacuum oven set to ˜80° C. to provide the desired compound.

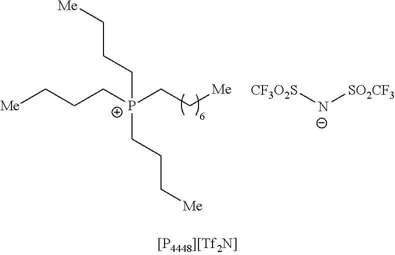

Tributyloctylphosphonium bis(trifluoromethanesulfonyl)imide [P₄₄₄₈][Tf₂N]

The synthesis of [P₄₄₄₈][Tf₂N] was conducted on a 10 mmol scale using tributyloctyl phosphonium bromide to provide 5.60 g (92% yield) of the title compound as a clear colorless oil. ¹H NMR (400 MHZ, CDCl₃) δ 2.20-2.02 (m, 8H), 1.62-1.38 (m, 16 H), 1.36-1.21 (m, 8 H), 0.97(t, J=8.0 Hz, 9 H), 0.88 (t, J=8.0 Hz, 3 H). ¹³C NMR (100 MHZ, CDCl₃) δ 119.9 (q, J=321.7Hz), 31.9, 30.9, 30.3, 30.1, 29.7, 29.4, 29.3, 28.8, 22.7, 22.3, 21.5, 18.9, 18.4, 13.9. ¹⁹F NMR (376 MHZ, CDCl₃) δ-78.85.

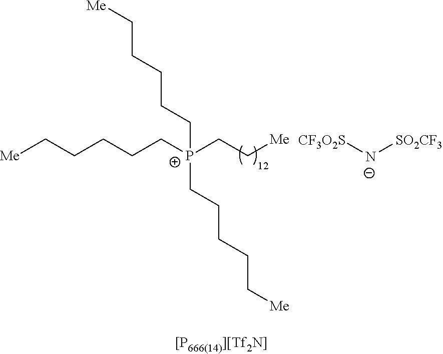

Trihexyltetradecylphosphonium bis(trifluoromethanesulfonyl)imide [P₆₈₆₁₄][Tf₂N]

The synthesis of [P₆₈₆₁₄][Tf₂N] was conducted on a 10 mmol scale using trihexyltetradecyl phosphonium bromide to provide 6.49 g (85% yield) of the title compound as a clear colorless oil. ¹H NMR (400 MHZ, CDCl₃) δ 2.17-2.01 (m, 8 H), 1.56-1.38 (m, 16 H), 1.37-1.18 (m, 32H), 0.88(t, J=7.1 Hz, 12 H). ¹³C NMR (100 MHZ, CDCl₃) δ 119.9 (q, J=321.6 Hz), 32.0, 30.9, 30.6, 30.5, 30.3, 30.1, 29.7, 29.7, 29.6, 29.5, 29.4, 29.3, 28.8, 22.7, 22.3, 21.5, 18.9, 18.4, 14.1, 13.9. ¹⁹F NMR (376 MHZ, CDCl₃) δ-78.82.

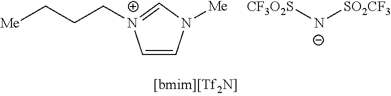

1-Butyl-3-methylimidazolium bis(trifluoromethanesulfonyl)imide [bmim][Tf₂N]

To a 10 mL round bottom flask equipped with a magnetic stir bar was added 1-butyl-3-methylimidazolium iodide (4.38 g, 20 mmol, 1 equiv.), placed under an atmosphere of N₂ then diluted with H₂O (5 mL). To the resulting solution was added LiTf₂N (6.03 g, 21 mmol, 1.05 equiv.) as a 6 M solution in H₂O. The mixture was stirred at rt for 15 hours then extracted with CH₂Cl₂ (3×15 mL). The combined organic fractions were washed with H₂O (3×10 mL), dried (MgSO₄), filtered, and concentrated under reduced pressure. The ionic liquid was then dried further for an additional 12 hours in a vacuum oven set to ˜80° C. to provide 8.14 g (97% yield) of the title compound as a faintly yellow oil. ¹H NMR (400 MHZ, CDCl₃) δ 8.73 (s, ¹H), 7.31 (s, 2 H), 4.17 (t, J=7.5 Hz, 2 H), 3.93 (s, 3 H), 1.85 (p, J=7.5 Hz, 2 H), 1.36 (h, J=7.4 Hz, 3 H), 0.96 (t, J=7.4Hz, 3 H). ¹³C NMR (100 MHZ, CDCl₃) δ 136.07, 123.67, 122.24, 119.79 (q, J=320.0 Hz), 49.94, 36.31, 31.92, 19.32, 13.20. 19F NMR (376 MHZ, CDCl₃) 0-79.11.

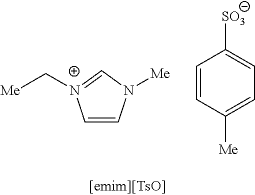

1-Ethyl-3-methylimidazolium tosylate [emim][TsO]

To a 50 mL round bottom flask equipped with a magnetic stir bar was added 1-ethyl-3-methylimidazolium chloride (1.47 g, 10 mmol, 1 equiv.), placed under an atmosphere of N₂ then diluted with MeCN (10 mL) and Et₂O (5 mL). To the resulting solution was added AgTsO (2.79 g, mmol, 1 equiv.). The mixture was stirred at rt for 1 hour then filtered through a bed of Celite to remove AgCl. The filter cake was washed with MeCN/Et₂O (1:1) (3×10 mL), then the filtrate was concentrated under reduced pressure. The crude oil was reconstituted in CH₂Cl₂ (50 mL) then washed with H₂O (3×10 mL), dried (MgSO₄), filtered, and concentrated under reduced pressure. The ionic liquid was then dried further for an additional 12 hours in a vacuum oven set to ˜80° C. to provide 2.74 g (97% yield) of the title compound as a yellow oil. ¹H NMR (400 MHZ, Acetone-d₆) δ 9.57 (s, ¹H), 7.78 (d, J=32.5 Hz, 2 H), 7.67 (d, J=8.2 Hz, 2 H), 7.13 (d, J=7.4 Hz, 2 H), 4.31 (q, J=7.3 Hz, 2 H), 3.97 (s, 3 H), 2.30 (s, 3 H), 1.44 (t, J=7.3 Hz, 3 H). ¹³C NMR (100 MHZ, Acetone-d₆) δ 147.2, 138.9, 138.3, 129.0, 126.7, 124.5, 122.9, 45.4, 36.4, 21.2, 15.8.

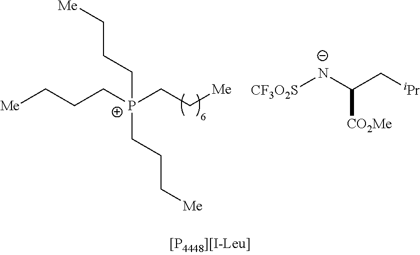

Tributyloctylphosphonium trifluoromethanesulfonylleucine methyl ester salt [P₄₄₄₈][I-Leu]

To a 20 mL round bottom flask equipped with a magnetic stir bar was added tributyl (octyl) phosphonium bromide (3.57 g, 9 mmol, 1 equiv.), placed under an atmosphere of N₂, then diluted with MeOH (9 mL). To the resulting solution was added 2 M methanolic KOH (9.1 mmol, 5.1 mL), precipitation of KBr was observed. The suspension was stirred at room temperature for 12 hours, then filtered through a bed of Celite to remove KBr. The filter cake washed with MeOH (3×5 mL) and to the filtrate was added N-trifluoromethanesulfonylleucine methyl ester (2.77 g, 9 mmol, 1 equiv.) and stirred at rt for 12 hours. The resulting solution was concentrated under reduced pressure and reconstituted in acetone (10 mL), filtered through a bed of Celite, then the resulted filtrate was concentrated under reduced pressure. The ionic liquid was then dried further for an additional 48 hours in a vacuum oven set to ˜80° C. to provide 3.90 g (75% yield) of the title compound as a yellow oil. ¹H NMR (400 MHZ, CDCl₃) δ 4.07 (dd, J=8.0, 5.8 Hz, 1 H), 3.66 (s, 3 H), 2.30 (dp, J=19.0, 6.5, 5.8 Hz, 7 H), 1.80 (dp, J=19.0, 6.5 Hz, ¹H), 1.67-1.39 (m, 18 H), 1.39-1.17 (m, 8 H), 0.97 (t, J=8.0 Hz, 9 H), 0.95-0.82 (m, 10H). ¹³C NMR (100 MHZ, CDCl₃) δ 175.4, 121.7 (q, J=326.0 Hz), 57.0, 51.5, 44.2, 30.8, 30.7, 29.0, 24.4, 24.0, 23.8, 23.7, 23.0, 22.6, 21.8, 18.9, 18.4, 14.1, 13.5. 19F NMR (376 MHZ, CDCl₃) δ-77.21. IR (neat) 2931, 1740, 1466, 1368, 1266, 1193, 1087, 912 cm⁻¹; HRMS (ESI) m/z: measured 315.318086 C₂₀H₄₄P+ (M) requires 315.317514, HRMS (ESI) m/z: measured 276.051640C₈H₁₃F₃NO₄S (M) requires 276.052287.

### DSE Experimental Screening of Candidate Ionic Liquids

The experimental procedure is briefly depicted in FIGS. 1A-1B. First, 2 mL of an IL and 2 mL of saline water with 3.7-5.0 wt % NaCl were mixed thoroughly at an elevated temperature (e.g., 45, 60 and 75° C.). The mixture was maintained at that temperature for ˜10 minutes for phase separation, expecting the brine to separate from the IL. The IL phase, which contained pure water dissolved within it, was collected, and cooled down to room temperature, precipitating out the fresh water due to lowered solubility in the IL. The separation of these two phases was facilitated by a centrifuge at 3000 rpm. The amount of recovered water was used to calculate the yield, and the water was also characterized for contents of NaCl ions and residual ILs to verify directional solubilities. The NaCl ion concentration was measured using Perkin Elmer Optima 8000 ICP-OES and the IL content is measured using Waters TQD triple mass spectrometer coupled to an Acquity ultrahigh pressure liquid chromatography system.

Concentration Measurement of NaCl, [Emim][Tf₂N], and [bmim][Tf₂N]

All Na⁺ concentration measurements were performed using Perkin Elmer Optima 8000 ICP-OES. Since all water samples in the experiment were deionized water, it was rational to assume that the molar concentration of Na⁺ was equal to that of NaCl. The Perkin Elmer Optima 8000 has a detection limit of ˜10 ppb, which was accurate enough to detect the concentration of NaCl in the recovered water and [emim][Tf₂N]. A set of calibration curves of conductivity versus Na⁺ ion concentration was created prior to the measurements. The detection of the concentration of [emim][Tf₂N] and [bmim][Tf₂N] in water was performed on the Waters TQD triple quadrupole mass spectrometer coupled to an Acquity ultrahigh pressure liquid chromatography system. Samples with different IL/water ratios are mixed in beaker with stirring speed of 300 rpm for 10 hours, and then a 3-hour sitting period was allowed to guarantee thermodynamic equilibrium of dissolution. Because the ILs tested have higher density than water, the solution in the top layer was extracted as the sample. The concentration of [Tf₂N]⁻ was measured using mass spectrometry to calculate the IL solubility in water. The intensity of a certain ion acquired by the mass spectrometer proportionally reflected the concentration of this kind of ion in the water solution.

### Ionic Liquids Tested

All the ILs studied are listed in Table 1. Performance information regarding the ILs tested to be less compatible with DSE are listed in Table 2 and the viscosities of the ILs are listed in

Table 3. The viscosity was measured with Discovery Hybrid Rheometer 2.

TABLE 1
General lonic Liquid Information

| Name | Abbreviation | Structure | Purity¹ | CAS No. |
|---|---|---|---|---|
| 1-Ethyl-3-methylimidazolium bis(trifluoromethylsulfonyl)imide | [emim][Tf₂N] | 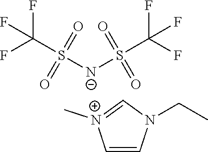 | >99% | 174899-82-2 |
| 1-Butyl-3-methylimidazolium bis(trifluoromethylsulfonyl)imide | [bmim][Tf₂N] | 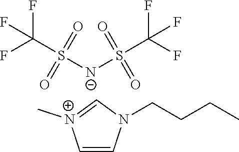 | >99% | 174899-83-3 |
| 1-Ethyl-3- methylimidazoliumtosylate | [emim][TsO] | 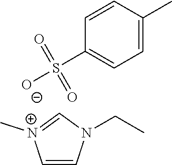 | >99% | 328090-25-1 |
| Tetrabutylphosphonium tosylate | [P₄₄₄₄][TsO] | 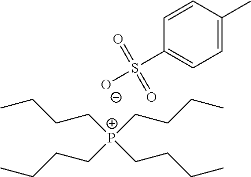 [SMILES: Cc1ccc(S(=O)(=O)[O-])cc1 ; CCCC[P+](CCCC)(CCCC)CCCC] | >99% | 116237-97-9 |
| Tetrabutylphosphonium bis(trifluoromethylsulfonyl)imide | [P₄₄₄₄][Tf₂N] | 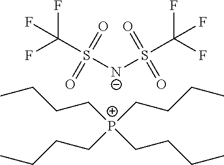 [SMILES: O=S(=O)([N-]S(=O)(=O)C(F)(F)F)C(F)(F)F ; CCCC[P+](CCCC)(CCCC)CCCC] | >99% | 210230-40-3 |
| Trihexyltetradecylphosphonium bis(trifluoromethanesulfonyl)imide | [P₆₆₆₁₄][Tf₂N] | 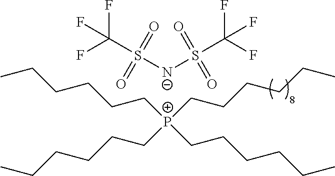 | >99% | 460092-03-9 |
| Tributyloctylphosphonium bis(trifluoromethanesulfonyl)imide | [P₄₄₄₈][Tf₂N] | 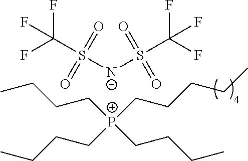 | >99% | |
| Tributyloctylphosphonium benzo[d]imidazol-1-ide | [P₄₄₄₈][Bzlm] | 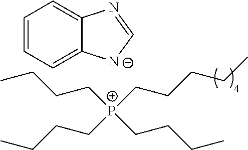 | >99% | |
| Tributyloctylphosphonium trifluoromethanesulfonylleucine | [P₄₄₄₈][I-Leu] | 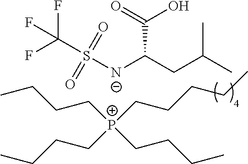 | >99% | |

¹Determined via ¹HNMR

TABLE 2
Task-Specific Ionic Liquid Directional
Solvent Extraction Performance

| Solvent | Performance |
|---|---|
| [emim][TsO] | Precipitate formation |
| [P₄₄₄₄][TsO] | Precipitate formation |
| [P₄₄₄₄][Tf₂N] | High melting temperature |
| | (~65° C.) |
| [P₆₆₆₁₄][Tf₂N] | High viscosity, low |
| | water yield (<0.012%/° C.) |
| [P₄₄₄₈][Tf₂N] | High viscosity, low |
| | water yield (<0.005%/° C.) |
| [P₄₄₄₈][Bzlm] | High viscosity |
| [P₄₄₄₈] I-Leu | High viscosity |

TABLE 3
Ionic Liquid Viscosity at 293 K

| Solvent | Viscosity in Pa · s |
|---|---|
| [emim][Tf₂N] | 0.033 ± 0.014 |
| [bmim][Tf₂N] | 0.056 ± 0.010 |
| [P₆₆₆₁₄][Tf₂N] | 0.081 ± 0.059 |
| [P₄₄₄₈][Tf₂N] | 0.433 ± 0.099 |
| [P₄₄₄₈][Bzlm] | 0.185 ± 0.092 |
| [P₄₄₄₈] I-Leu | 1.017 ± 0.078 |

The viscosities of [emim][Tf₂N], [bmim][Tf₂N] and [P₆₆₆₁₄][Tf₂N] are similar as those reported in the literature.

### Molecular Dynamics Simulation Details

The GROMACS (Groningen Machine for Chemical Simulations) package was used to perform MD simulation. The all-atom optimized potential for liquid simulation (OPLS-aa) force filed was used for [emim][Tf₂N]. All parameters and status settings used for the simulations are listed in Table 4. The TIP3P model and TIP4P model were separately used for water molecules to ensure that the calculated free energy tendencies were independent of force field (Table 5). In the simulations, a cutoff of 1.0 nm for van der Waals (vdW) and short-range electrostatic interaction was used. For the long-range electrostatic interactions, the Fast Particle-Mesh Ewald (PME) method with a 0.12 nm spacing for the fast-Fourier transformation (FFT) grid and a 6^{th} order interpolation scheme were used. All bonds were constrained by the Parallel Linear Constraint Solver (P-LINCS), and a time step of 2 fs was used for all simulations. Cubic simulation boxes with sides of ˜20 nm were built to guarantee that there is no interaction between the ion and its images in the periodic cells. The simulation of a dilute limit is empowered by this.

TABLE 4
Forcefield Parameters

| Parameter | Value/Status | |
|---|---|---|
| Coulomb type | PME | |
| Coulomb cut-off | 1.0 | nm |
| Periodic boundary conditions | All direction (xyz) | |
| Relative dielectric constant | infinity | |
| Cut-off distance for short- | 1.0 | nm |
| range neighbor list | | |
| Distance to start switching | 0.8 | nm |
| the Lennard-Jones Potential | | |
| The cut-off distance for the | 1.0 | nm |
| Lennard-Jones Potential | | |

| | |
|---|---|
| PME interpolation order | 6 |
| Dipole correction to the | off |
| Ewald summation | |
| Constraints | h-bonds |
| Neighbor searching type | grid |
| Soft-core alpha parameter | 0.5 |
| Power for lambda of soft- | 1 |
| core function | |
| Soft-core sigma for particles | 0.3 |
| having C6 or C12 parameter | |
| smaller than soft-core sigma | |

### Free Energy Calculation

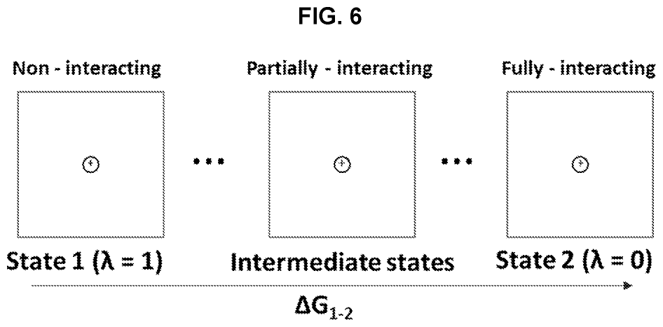

Thermodynamics integration was used with the coupling factor method for free energy calculation. In the coupling factor method, in order to calculate the free energy difference between two states, the Hamiltonian of a system, H, was manually changed by adjusting the coupling factor λ using the soft-core strategy. The free energy difference between two states, ΔG₁₋₂, can be calculated as:

$$Δ {G}_{1 - 2} = {\int }_{{λ}_{2}}^{{λ}_{1}} 〈 \frac{\partial H ( λ )}{\partial λ} 〉 d λ \qquad ( S1 )$$

where the angle bracket denotes ensemble average. In the free energy calculation using thermodynamic integration, an ion dissolution process was calculated by “appearing” the ion in the solvent through gradually switching on the non-bonded interactions, including vdW forces and electrostatic interactions, between the ions and the solvent molecules (FIG. 6). Appropriately formulating the λ-dependent non-bonded potential functions can conduct this process, with λ=1 corresponding to the non-interactions state and λ=0 corresponding to the fully interacting states. Around 30 discrete λ points are chosen between 1 and 0, and ∂H (λ)/∂λ is evaluated analytically in each MD simulation with different λ values. The detailed information of the λ-dependent potential functions can be found in Straatsma & McCammon, Ann. Rev. Phys. Chem. 43:407-35 (1992).

In each simulation, the system was first equilibrated for 20 picoseconds (ps) with a constant volume (NVT ensemble, Canonical Ensemble (NVT): a collection of all systems whose thermodynamic state is characterized by a fixed number of atoms, N, a fixed volume, V, and a fixed temperature, T), followed by a 100 ps second equilibration with constant pressure (NPT ensemble, constant-temperature, constant-pressure ensemble (NPT): allows control over both the temperature and pressure. The unit cell vectors are allowed to change, and the pressure is adjusted by adjusting the volume). Then, a 6 nanosecond (ns) production run in the NPT ensemble was performed, where the derivative, ∂H (λ)/∂λ, was evaluated and time was averaged. After collecting all the derivatives at each ∂H (λ)/∂λ point, trapezoidal numerical integrations were used to calculate the free energy difference using Eq. (S1). The temperature was set at 350 K for all simulations.

A common reference was needed to make a meaningful comparison between free energies. According to the definition, the solvation free energy of a solute molecule in a solvent is the value to describe the difference of the value between two states: the crystalline solid state and the dissolved state. To accurately determine the solvation free-energy value, two steps are needed: breaking the crystal lattice and dissolving the resulting gaseous ion. However, since the breaking of the crystal lattice step was identical in all simulations, and the purpose of the simulation was to compare the tendency of the solute molecules being dissolved in different solvents (e.g., in water versus in directional solvents), only the free energy change in the second step needed to be calculated. All values from the simulation are listed in Table 5.

TABLE 5
Free Energy of Solvation from
Simulation at 350 K (77° C.)

| | | Free Energy of |
|---|---|---|
| Solute | Solvent | Solvation (KJ/mol) |
| TIP4P | TIP4P | −22.9 |
| TIP4P | [emim][Tf₂N] | −26.5 |
| [emim][Tf₂N] | TIP4P | −23.3* |
| [emim][Tf₂N] | [emim][Tf₂N] | −38.6* |
| NaCl | TIP4P | −699.5 |
| NaCl | [emim][Tf₂N] | −677.7 |
| TIP3P | TIP3P | −23.9 |
| TIP3P | [emim][Tf₂N] | −24.0 |
| NaCl | TIP3P | −709.6 |
| [emim][Tf₂N] | TIP3P | −15.1* |

*Solvation free energy calculated involving [emim][Tf₂N] as a solute used ionic pair in vacuum (149.5 KJ/mol) as the reference state.

### Ternary Phase Simulation

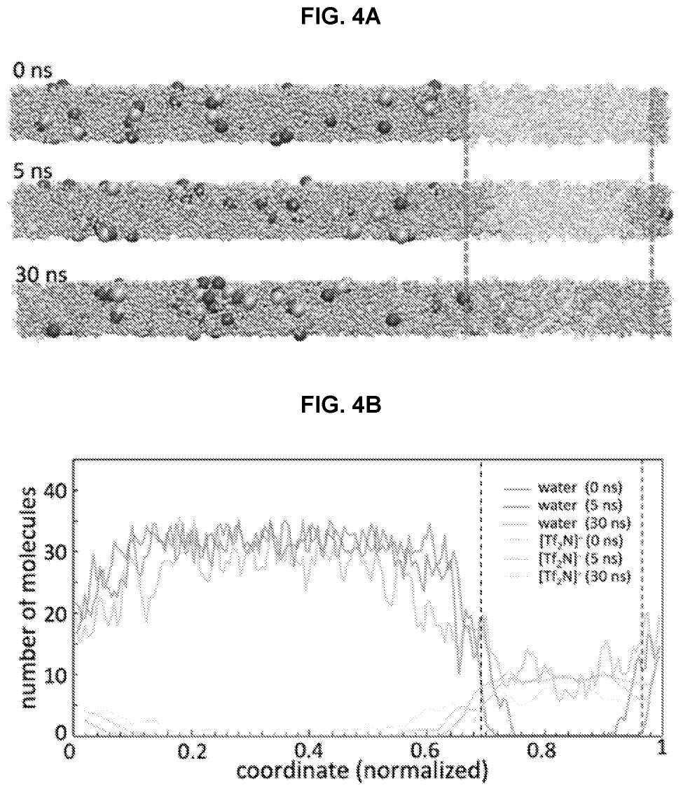

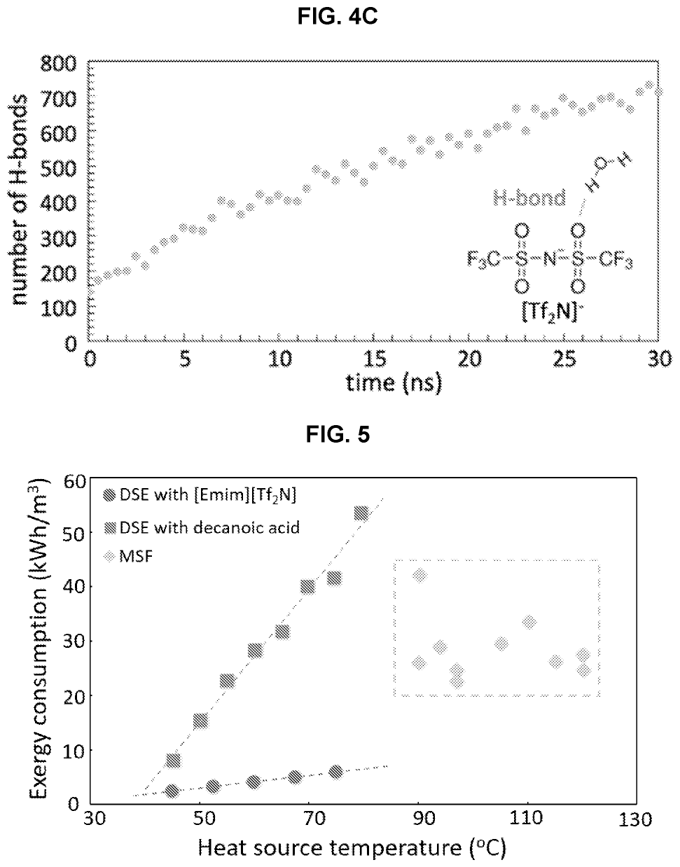

The procedure to set up the ternary phase simulation shown in FIG. 4 of the main text is as follows: (1) For saline water, 3.87% w/w of Na⁺ and C⁻ are dispersed into the water phase. (2) For [emim][Tf₂N]. the phase is performed in NPT ensemble. (3) NaCl solution and [emim][Tf₂N] are moved into the same box, and an equilibrium run is processed in the NPT ensemble to fully relax the structure at 350K. (6) A production run in NPT ensembles at 350K and 1 atm for 30 ns is carried out.

### Energy For Heat and Exergy Analysis

The procedure of Bajpayee et al., Energy Environ. Sci. 4:1672-1675 (2011) was used for the exergy calculation. The energy consumption was calculated based on the heat needed to increase the relevant mixture to the desired temperature. For a cycle between 20° C. (T₀) and a given top brine temperature (T_{TBT}), the thermal energy needed to heat the feed water-IL mixture to obtain a unit mass of produced fresh water is:

$${Q}_{lab} = ( \frac{{C}_{IL}}{{Y}_{w}} + {C}_{w} ) ( {T}_{T B T} - {T}_{0} ) \qquad ( S 2 )$$

where Y_{w}is freshwater yield, and C_{IL} and C_{W} are respectively the specific heat of IL and water. The data from Kabra et al., Proceedings 12th IEA Heat Pump Conference (2017) was used to calculate C_{IL} of [emim][Tf₂N]. In larger scale process, the heat recovery scheme can be implemented via heat exchangers to harvest thermal energy from the hot stream to pre-heat the fee water-IL mixture. If a practical heat exchanger efficiency (η_{HE}) of 0.8 is used, which is common in industrial applications, the net amount of thermal energy from the heat source to fuel the loop is:

$${Q}_{ind} = {Q}_{l a b} ( 1 - {η}_{H E} ) \qquad ( S 3 )$$

To highlight the merit of using low temperature thermal energy, the exergy consumption was calculated, which depends on the top brine temperature of the process:

$$E = {Q}_{ind} ( 1 - \frac{{T}_{0}}{{T}_{TBT}} ) \qquad ( S 4 )$$

Using Eqs. (S2) and (S3), Eq. (S4) can be re-written as

$$E = ( \frac{{C}_{IL}}{{Y}_{w}} + {C}_{w} ) ( 1 - {η}_{H E} ) ( {T}_{T B T} - {T}_{0} ) ( 1 - \frac{{T}_{0}}{{T}_{T B T}} ) \qquad ( S 5 )$$

To estimate the energy needed for nanofiltration process, calculations were used from Elimelech & Phillip, Science 333:712-717 (2011) and Cai et al., Environ. Sci.: Water Res. Technol. 1:341-347 (2015). The minimum energy required to separate solute from water is related to solution temperature, water activity and water recovery rate, as shown in the following equation:

$${W}_{min} = - R T \times ln ( {a}_{w} ) \times \frac{ln ( \frac{1}{1 - Y} )}{Y} \qquad ( S 6 )$$

where R, T, a_{w} is and Y respectively represent the gas constant, temperature, the water activity, and the water recovery rate. By combining Eq. (S6) with the definition of osmotic pressure (II),

$$Π = - RT \times \frac{ln ( {a}_{w} )}{{V}_{w}} \qquad ( S7 )$$

where V_{w} stands for the molar volume of water, one obtains:

$${W}_{min} = {V}_{w} \times Π \times \frac{ln ( \frac{1}{1 - Y} )}{Y} \qquad ( S8 )$$

If water recovery approaches zeros or if osmotic pressure is a constant value with water recovery, the theoretical minimum energy is:

$${W}_{0} = {V}_{w} \times Π \qquad ( S9 )$$

If water recovery reaches 98.5% as the ratio shown in Cai et al., Environ. Sci.: Water Res. Technol. 1:341-347 (2015), the theoretical minimum energy is:

$${W}_{0} = 4.27 {V}_{w} \times Π \qquad ( S10 )$$

### Pump Energy for Heat Exchanger

The methodology used is described in Alotaibi et al., Desalination 420:114-124 (2017). In order to maintain the running of the DSE desalination plant, three pumps are needed. The first pump is used for pumping the directional solvent from the low-temperature container to the high-temperature container, and the low-temperature directional solvent is heated in this process. The second is used for pushing the directional solvent from the high-temperature container to high-temperature container, and the high-temperature directional solvent is cooled in this process. The third pump is used for pumping the sea water into the system.

The exchanged heat in the heat exchanger can be expressed in the equation;

$$\dot{Q} = U \times A \times LMTD \qquad ( S11 )$$

In Equation (S11), \dot{Q} is the heat transfer rate, U is the heat transfer coefficient which is

$$\frac{1}{U} = \frac{1}{{h}_{w a t e r}} + \frac{{x}_{t h i c k n e s s}}{k} + \frac{1}{{h}_{ds}} \qquad ( S12 )$$

where h_{water} is the convective heat transfer coefficient for water, and h_{ds} is that for [emim][Tf₂N], and x_{thickness} is the thickness of the wall of the pipe. By setting the operation temperatures to 20° C. and 45° C. in the DSE cycle, the length of the pipe, L, can be calculated which is then used for pressure drop calculation below.
Pressure drop, Δp, can be calculated as:

$$Δ p = f \times ρ \times \frac{L}{{D}_{h}} \times \frac{{v}^{2}}{2} \qquad ( S13 )$$

where fis the friction factor, ρ is the density of the flowing liquid, v is the velocity of the flow, L is the length of the flow in the heat exchanger, and Dₕ is the hydraulic diameter of the flow. The friction factor can be calculated from Churchill's equation:

$$f = 8 ( {( \frac{8}{R e} )}^{12} + ( \frac{1}{{( {Θ}_{1} + {Θ}_{2} )}^{1.5}} ) ) \qquad ( S14 )$$

In equation S14,

$${Θ}_{1} = {[ - 2.457 ln ( {( \frac{7}{R e} )}^{0.9} + 0.27 \frac{ε}{D} ) ]}^{16}$$

and

$${Θ}_{2} = {( \frac{37530}{Re} )}^{16} ,$$

Re is the Reynolds number, and ε is the roughness of the surface. The following equation was used to estimate the pumping power needed:

$${\dot{P}}_{p} = \frac{\dot{V} Δ p}{{η}_{p} {η}_{e}} \qquad ( S15 )$$

where \dot{V} is the flow rate of volume and ηₚ, ηₑ stand for the pump efficiency and the electrical motor efficiency, respectively.

In the aforementioned calculations, it was assumed that all the cylinder tubes are made of copper. The inner diameter of the tube connected to pump 1 was 0.20 m, and the diameter of the tube connected to pump 2 and pump 3 was 0.20 m. The length of the tube connected to pump 1 was 15 meters (m), and the tube connected to pumps 2 and 3 had a total length of 15 m. Refer to Alotaibi et al., Desalination 420:114-124 (2017) for the indication of the pumps. The roughness of the tube was set to 0.34. All calculations were based on a freshwater production rate of 1 kg/s, which needs a flow rate of IL of ˜13.3 kg/s based on the water yield of the IL. With the pipe diameter of 0.20 m, the IL flow velocity was 0.28 m/s. The density of [emim][Tf₂N] was 1520 kg/m³. The thermal conductivity of [emim][Tf₂N] was taken from Ge et al., J. Chem. & Eng. Data 52:1819-1823 (2007). Because the convection heat transfer coefficient for [emim][Tf₂N], was unknown h_{ds}, the value of h_{water} was used as the value of h_{ds}, which was believed not to change the order of magnitude for the calculation.

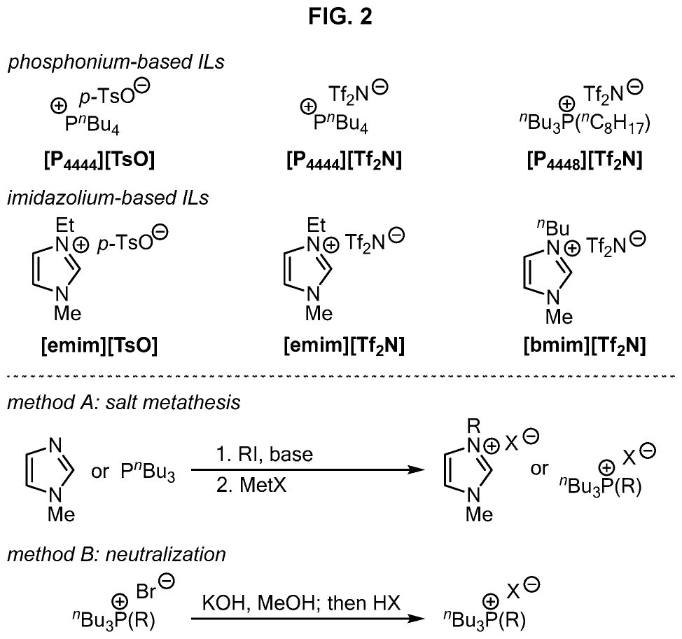

This study examined a series of sulfonate anion-based TSILs as DSs for DSE desalination. The p-toluene sulfonate anion salts of tetrabutylphosphonium IL [P₄₄₄₄][TsO] were initially targeted, and the corresponding 1-ethyl-3-methylimidazolium-based IL [emim][TsO] was also synthesized due to the well-established physical attributes of the imidazolium ILs (FIG. 2). Similarly, the imide anion-based ILs [P₄₄₄₄][Tf₂N] and [emim][Tf₂N] were assembled in an effort to evaluate the relatively non-polar characteristics of the bistriflimide component. To determine the impact of changes in IL hydrophobicity, density, and viscosity on DSE performance, two different alkyl chain lengths were incorporated in the phosphonium and imidazolium cations to provide the unsymmetrical ILs [P₄₄₄₈][Tf₂N] and [bmim][Tf₂N]. Each targeted IL was readily synthesized using a salt metathesis approach wherein alkylation of either the parent N-methyl imidazole or PⁿBu₃ with the desired alkyl iodide or bromide followed by anion exchange to incorporate the desired sulfonate or bistriflimide anions. Alternatively, treatment of the tetraalkyl phosphonium bromide with KOH in MeOH followed by exposure to the sulfonic acid or triflimide provided the desired phosphonium ILs. Each IL was obtained in excellent yield and evaluated using ion exchange chromatography and Karl-Fisher titration for residual halide and water content, respectively.

The DS performance in a DSE desalination cycle was evaluated for the assembled ILs, which involved first exposure to water bearing 3.7-5.0 wt % of NaCl (11,000-15,000 ppm of Na⁺) followed by thorough mixing at elevated temperatures of 45, 60 or 75° C. The mixture was held for ˜10 mins at each stage to allow for complete phase separation. For the strongest performing IL identified, [emim][Tf₂N], a settling time as low as 2-min was found to be sufficient. The water-enriched IL phase was then removed and allowed to cool to room temperature, which resulted in a second separation of the IL and desalinated water. The amount of residual NaCl and IL in the recovered aqueous phase was then measured using atomic emission spectroscopy and liquid chromatography-mass spectrometry.

Of the characterized ILs, two were identified as potentially viable frameworks for DSE, and their DSE-relevant solubilities are shown in Table 1 with decanoic acid as the comparable standard. Those ILs not shown underwent an undesirable salt metathesis with saline water, exhibited a prohibitively high melting temperature. or exceptionally high viscosity (see Table 2). While the imidazolium IL [emim][TsO] also induced a salt metathesis reaction, the bistriflimide [emim][Tf₂N] exhibited exceptional ion rejection at >96% with low solubility in water in the range of 130-150 ppm and a surprisingly high-water yield of 0.3%/C (Table 6). In contrast. [bmim][Tf₂N] provided a lower water yield of <0.082%/° C. despite a reasonable ion rejection rate of ˜70% and favorable solubility in water (<90 ppm).

TABLE 6
Task-Specific Ionic Liquid Directional Solvent Extraction
DesalinationPerformance ^{a}

| | | Solubility in | H₂O Yield |
|---|---|---|---|
| IL | Ion Rejection | H₂O (ppm) | (%/° C.) |
| [bmim][Tf₂N] | 70.5% ± 2.9% | <90 | <0.082 |
| [emim][Tf₂N] | 97.5% ± 0.8% | 130-150 | 0.304 ± 0.023 |
| decanoic acid | 96.9%~98.0% | 36-150 | 0.025 ± 0.002 |

^{a} [bmim][Tf₂N] and decanoic acid group evaluation was performed at 75° C. [emim][Tf₂N] evaluation was performed at 45, 60, and 75° C.

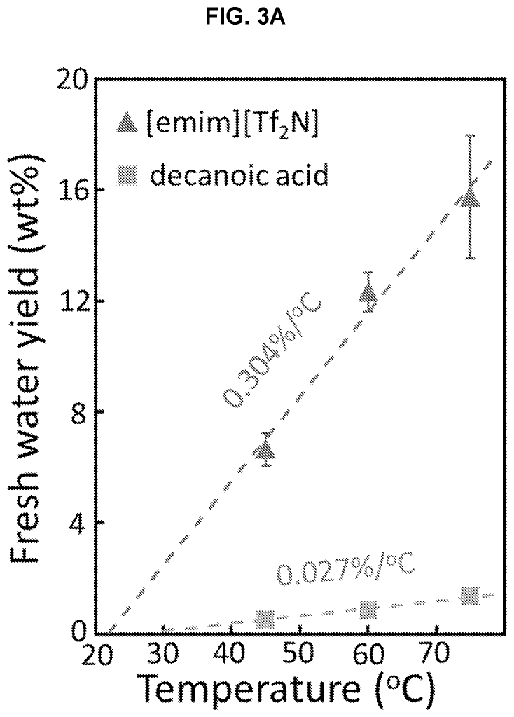

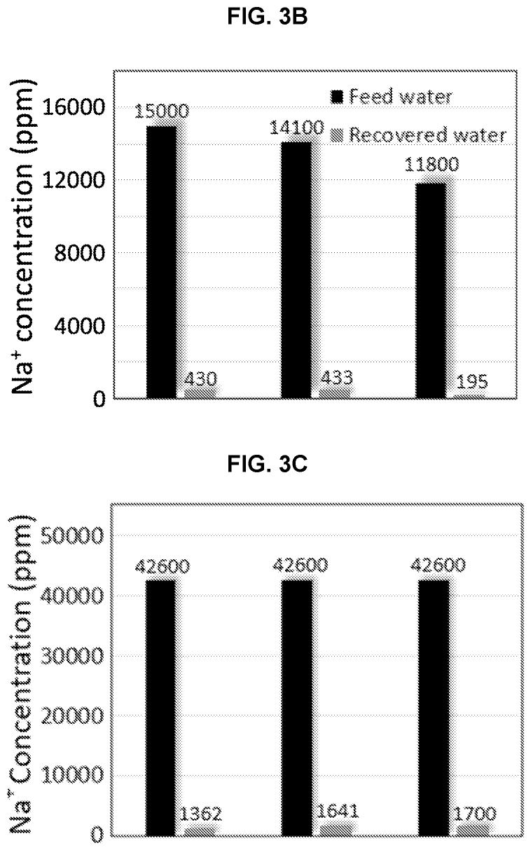

Given the promising ion rejection rate, water solubility, and water yield exhibited by [emim][Tf₂N], the fresh_{water} recovery of this IL was examined at elevated temperatures. Exposure of [emim][Tf₂N] to a 3.7 wt % NaCl saline feed at 45, 60, and 75° C. revealed an average water yield of 0.3±0.023%/° C. (FIG. 3A). As is evident, the exceptional water yield displayed by [emim][Tf₂N] in comparison to decanoic acid supports the supposition that IL-based DSs constitute a viable class of soft materials toward the development of more efficient DSE desalination process.

For saline with NaCl salinity in the range of 3.0-3.8 wt % (11,000-15,000 ppm of Na⁺), [emim][Tf₂N] exhibited outstanding ion rejection rates of 97.0-98.3% (FIG. 3B), bringing the salt content below the drinking water standard of 500 ppm. To test the capacity of [emim][Tf₂N] as a DS to treat high salinity water, several DSE cycle experiments were conducted using 10.8 wt % aqueous NaCl solution (42,600 ppm of Na⁺) as the feed saline. An ion rejection rate of 96.0-96.8% was observed even when [emim][Tf₂N] was exposed to high concentrations of aqueous NaCl. These results indicate that [emim][Tf₂N] exhibits promising physical attributes to conduct desalination of water at both moderate and high salinity. Experiments with saturated NaCl used as feed saline were also performed and indicate that desalination can be performed using DSE with [emim][Tf₂N]. The ion rejection rate of the DSE cycle reaches 96.5% and the fresh_{water} yield indeed drops to 0.157%/° C. for this compound. The reduction can be understood because higher salinity water is more thermodynamically stable due to the ion-water electrostatic interaction and more difficult to extract, which was previously examined and also observed for decanoic acid. However, the water yield of IL for saturated saline is still 5.8-times of that of decanoic acid treating 3.8% NaCl feed water (0.027%/° C.).

Additionally, the water yield of [emim][Tf₂N] is consistent over a temperature range of 45-75° C., which suggests that low quality waste heat may be suitable to power this desalination process. These DSE experiments have been repeated for several cycles by re-using the [emim][Tf₂N], and no performance degradation in ion-rejection or water yield was observed. This confirms that ion removal using [emim][Tf₂N] in DSE is not due to the IL absorbing NaCl ions from saline water but caused by IL rejecting these ions. Additional experiments were performed to measure IL concentration in the brine with different salinities. In these experiments, the brine (MX, FIG. 1) salinity is 4% or higher depending on the initial mixing ratio of the saline water and IL. For the MX with 4% salinity, the tested IL concentration is 4 ppm. The IL solubility in the saturated NaCl solution is further lower than 4 ppm. As a result, it was concluded that the IL residue in the MX is at a very low level. This is understandable as existing ions in water make the dissolution of additional ions more difficult.

The ion residue in IL, measured by the Nat concentration, was ˜50 ppm. Importantly, DSE was performed for 30 cycles using the same IL and the salt concentration in IL has been steady and the desalination performance was not degraded. As a result, the ion residue in the IL was believed to have reached a steady state and does not influence the desalination performance.

Molecular dynamics (MD) simulations were employed in combination with thermodynamic integration to calculate the solvation free energies at room temperature for NaCl, water, and [emim][Tf₂N]. In brief, a solute molecule (e.g., NaCl or H₂O) was simulated to “gradually appear” into the solution, and free energy needed for this process is calculated as the solvation free energy (see FIG. 6). The values from the calculation are for solvation from a vacuum state to the solution state, where the vacuum state serves as the common reference for comparing the thermodynamic stability of the solute molecule in different solvents. All calculations were performed at 350 K.

By comparing the solvation free energies, thermodynamic stability was determined in the respective environments for each solute molecule, which ultimately aided the understanding of the variable solvation tendencies. The calculated solvation free energy of NaCl in [emim][Tf₂N] is −677.7 KJ/mol, which is in contrast to −699.5 KJ/mol found for NaCl in water. This indicates that the NaCl salt favors solvation in the aqueous media over the corresponding IL phase and rationalizes the observed ion rejection capability of [emim][Tf₂N] in the DSE process. Similarly, the solvation free energy of water in [emim][Tf₂N] of −26.5 KJ/mol was lower than that of water in water (−22.9 KJ/mol), which is consistent with the observed propensity for water to dissolve into the IL. Additionally, the calculated solvation free energy of −38.6 KJ/mol for [emim][Tf₂N] in itself was less than that of [emim][Tf₂N] in water (−23.3 KJ/mol), which suggested that it is thermodynamically unfavorable for the IL to dissolve in water. The above two cases related to [emim][Tf₂N] solvation, using the state of a [emim][Tf₂N] ionic pair in vacuum as the reference level. Overall, these calculations were consistent with the experimental observations, revealing that [emim][Tf₂N] displays favorable DS thermodynamic properties of water insolubility while concurrently capable of solvating water molecules and rejecting salt ions. The above simulations used the TIP4P as the water model. The TIP3P water model was also used, and the same solvation tendencies were obtained (see Table 5).

A simulation of 3.7% NaCl water solution in contact with [emim][Tf₂N] was also run at 350 K for a duration of 30 ns. FIG. 4A shows snapshots of the ternary system simulation. Throughout the simulation, almost all Na⁺ and Cl⁻ remained in the water phase with only two of them appeared to have diffused into the [emim][Tf₂N] phase. A large number of water molecules diffused into the [emim][Tf₂N] phase, but only limited number of [emim]⁺ and [Tf₂N]⁻ ions diffused into the water phase. FIG. 4B shows the density profiles of water and IL at different times corresponding to the snapshots in FIG. 4A. It was apparent that water diffusion into IL is much more significant than IL diffusion into water. These phenomena generally agree with the solvation free energy calculation results and experimental observations. The bonding nature between water and IL molecules was alos analyzed and hydrogen bonds were found to exist between water and the [Tf₂N]⁻ ions (FIG. 4C). The hydrogen bonds were formed between the water molecules and the polar sulfonyl groups of the [Tf₂N] ions (see inset in FIG. 4C), and the number of hydrogen bonds grew as more water molecules dissolved into IL.

The ability to operate a DSE process at the relatively mild temperatures shown with [emim][Tf₂N] (e.g., 45° C.) constitutes a significant advantage wherein waste heat is a viable source of operating power. Exergy is the maximum amount of extractable work from a heat source with Carnot efficiency and is a useful measure of the overall system efficiency when comparing different desalination technologies. As shown in FIG. 5, exergy consumptions of DSE with [emim][Tf₂N] and decanoic acid both increased with increasing heat source temperature. A DSE process operated with [emim][Tf₂N] has an exergy cost of 2.4 kWh/m³ at 45° C. and 5.9 kWh/m³ at 75° C. The pumping power requirement is 0.31 kWh/m³ at 45° C., and this value will be smaller at higher temperature, according to Alotaibi et al., Desalination 420, 114-124 (2017).

Comparing the exergy consumptions of DSE processes revealed a staggering reduction in exergy cost by 70% at 45° C. when [emim][Tf₂N] was employed as the DS in contrast to decanoic acid. Additionally, this observed exergy penalty reduction increased to 89% at 75° C. Exergy consumption of a state-of-the-art thermal desalination technolog—-multi-stage flash (MSF) is also provided for comparison. Besides the advantage of utilizing lower temperature (45-75° C.) heat sources compared to MSF (>85° C.), the exergy consumption of DSE using [emim][Tf₂N] was significantly lower than that of the MSF. While decanoic acid exhibited comparable performance advantages over MSF, these are only realized at operating temperatures below ˜55° C. (FIG. 5). Elimelech & Phillip, Science 333:712-717 (2011) reported that an energy consumption as low as 2 KWh/m³ from new large-scale reverse osmosis (RO) plants might be achieved. In addition, Voutchkov, Desalination 431:2-14 (2018) showed that in the state-of-the-art RO plants, the energy cost related to the RO process alone, excluding those from processes like pre-treatment and water delivery, is 2.54 kWh/m³. As a result, DSE has similar energy costs as RO when operating at low temperature, let alone its unique capability of harvesting low temperature waste heat. Moreover, DSE is suitable for small scale applications, especially for low-resource settings, where the energy cost for small-scale RO is high (up to 17 kWh/m³), largely due to the lack of centralized infrastructures for mechanical energy recovery.

While residual [emim][Tf₂N] in the recovered water should be low (130-150 ppm), there is no established toxicity standard for this IL in drinking water. To completely remove and recover all IL from the produced water, nanofiltration (NF) can be used as implemented by Cai et al. Environ. Sci.: Water Res. Technol. 1:341-347 (2015) and Elimelech and Phillip, Science 333:712-717 (2011) for their IL-FO desalination for similar residue removal purposes. This will also eliminate the loss of IL during operation. The exergy cost for the NF process is a mere 0.011KWh/m³ given the low osmotic pressure associated with a minimal concentration of residual IL, thereby avoiding a significant energy penalty.

The efficacy of IL-based DSs appeared to be modulated dramatically by structural perturbations of the IL. The optimal IL discovered being [emim][Tf₂N] contains small alkyl chains adorned on the imidazolium cation and a hydrophobic resonance stabilized bistriflimide anion. Conversely, the ILs which failed to provide DSE behavior contain structural features altering hydrophobicity, size, and charge dispersion of the anion and cation. As in the case of [emim][TsO], which contains the larger [TsO]⁻ anion in comparison to [emim][Tf₂N], a precipitate was observed upon subjection to a DSE cycle, presumably a mixture of [emim][CI] and [Na][TsO].

The [bmim][Tf₂N] IL while providing moderate DSE behavior, failed to reject NaCl (˜70%) as efficiently as [emim][Tf₂N] or decanoic acid and has lower solubility in water (<90 ppm) as well as water yield (0.082%/° C.), likely due to the lengthened aliphatic chain. The [bmim]⁺ cation had an increased hydrophobicity due to increased chain length as well as a weaker cation-anion interaction as longer aliphatic chain lengths present larger steric interaction with the [Tf₂N]⁻ anion. Therefore, a stronger interaction may be achieved between [bmim][Cl] and [Na][Tf₂N] due to the relative size differences yielding more intimate ion pairs sequestering more NaCl. As a result, the salt rejection rate is only around 70% according to the results. The [bmim][Tf₂N] IL, however, might be used for treating low salinity water (<1600 ppm), where the 70% ion rejection rate can bring the salinity down to meet the drinking water standard of 500 ppm. The increased hydrophobicity of the [bmim] +cation compared to the [emim]⁺ cation can also explain its comparatively lower water yield. MD simulations were preformed to calculate the free energy of water in [bmim][Tf₂N], and the result (−24.3 KJ/mol) is indeed higher than that of water in [emim][Tf₂N] (−26.5 KJ/mol).

In comparison to the imidazolium-based cations whose positive charge is resonated into the aromatic system, the tetraalkyl phosphonium cations have a point charge dispersed over a smaller area. This difference in polarization alters ion-pair strength, which may be responsible for the decrease in the desired DS behavior of tetralkyl phosphonium ILs, where larger charge separation weakens the interion interactions of the IL, allowing large amounts of sodium chloride to penetrate into the IL phase leading to undesired metathesis reactions. In addition to size and charge distribution, the viscosity of the IL affects the DSE behavior. For example, the ion rejection rate is found to be very low for [P₄₄₄₈][Tf₂N] whose viscosity is 0.433 Pa·s, which is very high (see Table 4). The concentration of recovered water is even higher than the feed water. Furthermore, high viscosity ILs are not optimal for practical applications as a large pumping power would be required to perform a DSE cycle.

In summary, DSE can be effectively used to extract fresh water from saline sources, even saturated saline water, by using [emim][Tf₂N]. The DSE technique, which can utilize the low temperature heat, can potentially result in low resource setting applications. By analyzing the results of several tested ILs and their DSE behavior, the chemistry-property relation was rationalized, which may be helpful for further identification of task specific DS ILs. This work is also expected to further stimulate research around the DSE technology, especially the exploration of even higher-performance DSs. To move DSE further closer to practical deployment, mass production of the identified ILs, process optimization, thermal system design with the potential integration of waste heat or renewable energy are topics worth additional research or engineering.

### Example 2

Synthesis and Characterization of lonic Liquids

All organic solvents were distilled under and argon atmosphere and passed through a column of molecular sieves prior to use. Deionized water was used for all reactions unless otherwise stated. Reagents were used as received by commercial sources without further purification. All reactions were carried out in oven dried glassware under nitrogen or argon at room temperature unless otherwise specified. Compounds were characterized by ¹H nuclear magnetic resonance (NMR) spectra obtained at 400 or 500 MHZ, ¹³C NMR obtained at 100 or 125 MHz, and ¹⁹F NMR obtained at 376 MHz. Chemical shifts are reported in parts per million (ppm, δ), and referenced from the solvent. Coupling constants are reported in Hertz (Hz). Spectral splitting patterns are designated as s, singlet; d, doublet; t, triplet; q, quartet; m, multiplet; comp, complex; app, apparent; and br, broad. Infrared (IR) spectra were obtained using a Thermo Electron Nicolet 380 FT-IR using a silicon (Si) crystal in an attenuated total reflectance (ATR) tower and reported as wavenumbers (cm⁻¹). High-and Low-resolution electrospray ionization (ESI) measurements were made with a JEOL JMS-AX505HA mass spectrometer.

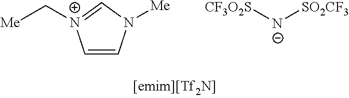

1-Ethyl-3-methylimidazolium bis(trifluoromethylsulfonyl)imide [emim][Tf₂N] was synthesized by reported methods. See e.g., Srour et al., Green Chem. 15:1341-1347 (2013).

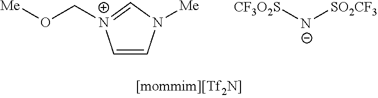

1-(methoxymethyl)-3-methyl-¹H-imidazol-3-ium bis ((trifluoromethyl) sulfonyl) amide ([mommim][Tf₂N])

To a 25 mL round bottom flask equipped with a magnetic stir bar under an atmosphere of N₂ was added 1-(methoxymethyl)-3-methyl-¹H-imidazol-3-ium bromide (1.04 g, 5 mmol) in acetone (5 mL). Then lithium bistriflimide (1.44 g, 5 mmol, 1 equiv.) was added and the mixture was stirred at rt for 3 d. The resulting solution was concentrated under reduced pressure and the residue was extracted with DCM (3×10 mL), dried (MgSO₄), filtered and concentrated under reduced pressure. The ionic liquid was then dried further for an additional 48 h in a vacuum oven set to ˜80° C. to provide 1.75 g (86%) of the title compound as a pale yellow oil. Notably, a secondary aggregate species was observed in CDCl₃ ([mom-mim][Tf₂N]_{aggregate}). [mom-mim][Tf₂N]ₘₐᵢₙ: ¹H NMR (500 MHZ, D₂O): δ 8.91 (s, 1 H), 7.59 (s, 1 H), 7.49 (s, 1 H), 5.51 (s, 2H), 3.91 (s, 3 H), 3.38 (s, 3 H); ¹³C NMR (125 MHZ, D₂O) δ 136.7, 124.3, 121.9, 119.4 (q. J_{F-C}=319.7 Hz), 80.0, 57.1, 36.1; 19F NMR (471 MHZ, D₂O): δ-82.4; 1H NMR (500 MHZ, CDCl₃): δ 8.98 (s, ¹H), 7.41 (t, J=1.8 Hz, 1 H), 7.32 (t, J=1.8 Hz, 1 H), 5.50 (s, 2 H), 4.01 (s, 3 H), 3.43 (s, 3 H); ¹³C NMR (125 MHz, CDCl₃) · 136.0, 124.1, 121.4, 119.7 (q, J_{F-C}=320.6 Hz), 80.8, 57.9, 36.8; 19F NMR (471 MHZ, CDCl₃): δ-82.3; [mom-mim][Tf₂N]_{aggregate}: ¹H NMR (500 MHZ, CDCl₃): δ 8.64 (br s, ¹H), 7.45 (br s, 1 H), 7.38 (br s, 1 H), 5.43 (br s, 2 H), 3.91 (br s, 3 H), 3.36 (s, 3 H); 13C NMR (125 MHZ, CDCl₃) δ 136.8, 124.4, 121.8, 119.7 (br q, J_{F-C}=320.9 Hz), 80.5, 57.4, 36.3; 19F NMR (471 MHZ, CDCl₃): δ-82.9; IR (neat): 3157.2, 3117.2, 2966.8, 1578.8, 1560.2, 1465.0, 1452.3, 1346.9, 1179.5, 1049.3, 920.3, 858.0, 791.2, 739.9, 609.3, 569.4, 509.5 cm⁻¹; HRMS (ESI) m/z: measured 127.0904 C₆H₁₁N₂O⁺ (M) requires 127.0866, HRMS (ESI) m/z: measured 534.0913 C₁₄H₂₂F₆N₅O₆S₂ (M+IL) requires 534.0910.

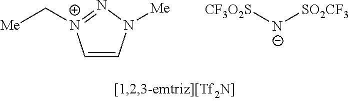

1-ethyl-3-methyl-1,2,3-triazolium bistriflimide ([1,2,3-emtriz][Tf₂N])

Triazole (145 mmol, 10.00 g), potassium carbonate (217 mmol, 40.00 g), and tetrahydrofuran (150 mL) were added to a round-bottom flask equipped with a stir bar and placed under an inert atmosphere. The reaction vessel was then cooled to 0-5° C. in and allowed to stir. lodomethane (217 mmol, 13.58 mL) was then added drop dropwise. After 24 hours of stirring at room temperature, the reaction mixture was cooled to −20° C. for approximately 1 h. The solids were then removed via suction filtration and the filtrate was concentrated in vacuo to afford 9.84g of 1-methyltriazole (82% yield). Methyl triazole (118 mmol, 9.84 g) and acetonitrile (150 mL) were added to a round-bottom flask equipped with a magnetic stir bar. While stirring at room temperature, bromoethane (177 mmol, 13.2 mL) was added dropwise. The reaction was then placed under argon, outfitted with a reflux condenser, and allowed to stir at (80° C.). After approximately 72 h, the reaction was cooled, and an additional 3 mL of bromoethane were added. The reaction of was then stirred at reflux for an additional 24 h. Acetonitrile was removed under vacuum and the liquid was filtered through a small plug of cotton. Upon filtration, the liquid solidified. 7.11 g of 1-ethyl-3-methyl-1,2,3-triazolium were recovered as a brown solid: ¹H NMR (400 MHZ, CDCl₃) δ 9.79-9.64 (m, 2H), 4.87 (q, J=7.4 Hz, 2 H), 4.56 (d. J=1.5 Hz, 3 H), 1.80-1.63 (m, 3 H). 1-Ethyl-3-methyl-1,2,3-triazolium bromide (56.2 mmol, 10.74 g), lithium bistriflimide (56.2 mmol, 16.14 g), and acetonitrile (60 mL) were added to a round-bottom flask equipped with a magnetic stir bar. The reaction was placed under argon gas and stirred at room temperature for approximately 72 h. Acetonitrile was removed under vacuum, and the crude product was reconstituted in a minimal amount of dichloromethane. Upon contact with dichloromethane, a white precipitate formed. The precipitate was removed via suction filtration. The filtrate was washed with H₂O (4×) and dried (MgSO₄) to afford 1-ethyl-3-methyl-1,2,3-triazolium bistriflimide: ¹H NMR (500 MHZ, CD₃CN) δ 8.31 (d, J=1.5 Hz, 1 H), 8.27 (d, J=1.5 Hz. 1 H), 4.59 (q, J=7.4 Hz, 2 H). 4.25 (s, 3 H), 1.59 (t, J=7.3 Hz, 3 H); ¹⁹F NMR (471 MHZ, CD₃CN) δ-83.37. ¹³C NMR (126 MHz. CD₃CN) δ 131.47, 130.17, 49.55, 13.86; HRMS+ m/z 112.0880 [C₅H₁₀N₃ requires 112.0869], 504.0908 [C₁₂H₂₀F₆N₇O₄S₂ requires 504.0917].

Hypersaline Desalination using lonic Liquids and Low-Temperature Heat

Previous work indicates that as a working IL, [emim][Tf₂N] shows good efficiency, both in two aspects, water yield (0.304%/° C.) and rejection rate (97.5% NaCl rejection rate). Previous studies have indicated that the primary reasons causing an IL's failure in DSE include, but are not limited to, the high viscosity and precipitate formation. Excessively high viscosity and precipitate formation obstructs the IL from being a viable DSE working substance. Based on the result of [emim][Tf₂N], a more thorough understanding of the IL structure-DSE performance relationship was desired. Several ILs with different targets were synthesized and evaluated (e.g., probed anion behavior, cation behavior, viscosity, etc.).

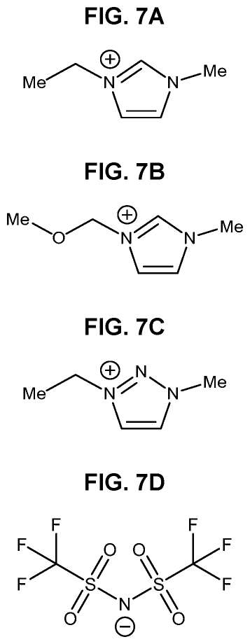

In view of the strong performance of the IL [emim][Tf₂N], the IL [mommim][Tf₂N] was synthesized to evaluate cations containing ethereal side chains and to probe IL viscosity. The IL [1,2,3-emtriz][Tf₂N] was also synthesized to probe role of 5-membered nitrogenated heterocyclic cations. The structures of [emim]⁺, [mommim]⁺ and [1,2,3-emtriz]⁺ are shown in FIG. 7 and the ion rejection rate, solubility of ILs in water, and water yield are listed in Tables 7-8

The [mommim][Tf₂N] viscosity was 0.056 Pa·s and the [1,2,3-emtriz][Tf₂N] was 0.035 Pa·s, as measured using TA Instrument Discovery HR-2. In the desalination process with hypersaline, [mommim][Tf₂N] exhibited strong DSE performance. For [mommim][Tf₂N], a 0.032%/° C. water yield was obtained with the NaCl (19.6 wt %, Na⁺ 77,300 ppm) feeding hypersaline in the temperature range from 20° C. to 65° C. Because the ion-water electrostatic interaction makes water in higher salinity saline more thermodynamically stable and increases the difficulty to attract. The average salt rejection rate in the samples was 89.5%, and the highest rejection rate that was obtained was 95.8%. Besides the experiment with hypersaline, an experiment with [mommim][Tf₂N] was performed with saline having a Na⁺ concentration similar to that of seawater (3.5 wt %, 14,000 ppm Na⁺). The recovered concentration 926 ppm was recovered from feeding saline with Na⁺ concentration 13700 ppm. Due to the weakened ion-water electrostatics interaction, the water yield obtained for [mommim][Tf₂N] was 0.24%/° C.

The effect of [1,2,3-emtriz][Tf₂N] with saline having a Na⁺ concentration similar to that of seawater was also tested. The rejection rate was 92.5%. The water yield in this saline concentration with [1,2,3-emtriz][Tf₂N] was 0.19%/° C. The solubility of [mommim][Tf₂N] in water was 270 ppm. The solubility of [1,2,3-emtriz][Tf₂N] was 390 ppm. A further experiment is also performed to test the solubility of these two ILs in 4% NaCl solution, and the solubility is one order of magnitude lower than the solubility in water.

The solubility of [mommim][Tf₂N] in 4% NaCl solution was about 26 ppm, and the solubility of [1,2,3-emtriz][Tf₂N] in 4% NaCl solution was about 41 ppm. The solubility of these two ILs in hypersaline is smaller than the solubility in 4% NaCl, respectively. The Na⁺ residual in the ILs is also lower than that in the water, which implies that Nat is rejected but not “absorbed” by the ILs.

TABLE 7
Comparison of Ion Rejection, Water Solubility, and Water
Yield for Seawater Salinity Feedwater

| | Ion | | | | |
|---|---|---|---|---|---|
| | rejection, | | H₂O | | |
| | seawater | | yield | Na+ | |
| | concen- | Solubility | (%/ | residual | |
| IL name | tration | in H₂O | ° C.) | in IL | Motivation |
| [emim] | 97.5% ± | 130 ppm- | 0.304 ± | ~50 ppm | |
| [Tf₂N] | 0.8% | 150 ppm | 0.023 | | |
| [mommim] | 95.2% | ~270 | 0.24%x | ~520 ppm | Ethereal |
| [Tf₂N] | | ppm | | | side chains/ |
| | | | | | viscosity |
| [1,2,3- | 92.5% | ~390 | 0.19% | ~330 ppm | Nitrogenated |
| emtriz] | | ppm | | | heterocyclic |
| [Tf₂N] | | | | | cations |

TABLE 8
Comparison of Ion Rejection, Water Yield, Water
Solubility, and Viscosity for Hypersaline Feedwater

| | Ion | H₂O yield | Solubility in | |
|---|---|---|---|---|
| | rejection, | (%/° C.) | saline, seawater | |
| IL name | hypersaline | hypersaline | concentration | Viscosity |
| [mommim] | 89.5% | 0.032 | 26 | 0.056 |
| [Tf₂N] | | | | Pa · s |
| [1,2,3- | | | 41 | 0.035 |
| emtriz] | | | | Pa · s |
| [Tf₂N] | | | | |

From the experiment result, the three ILs, [emim][Tf₂N], [mommim][Tf₂N]. and [1,2,3-emtriz][Tf₂N] were not only are able to purify saline with seawater salinity, but also treated hypersaline.

Employing molecular dynamics (MD) simulations provided a molecule-level understanding of the experimentally observed directional solubilities by calculating the solvation free energies in different solute and solvents cases. Previously, MD simulation was used in the fatty acid DSE cases, and the MD calculation was consistent with the experiment result. Besides fatty acids, MD simulation of [emim][Tf₂N] was also accordant with experiment result. Briefly, a solute molecule was simulated to “gradually appear” into the solution, and free energy need in this process is calculated to be the solvation free energy.

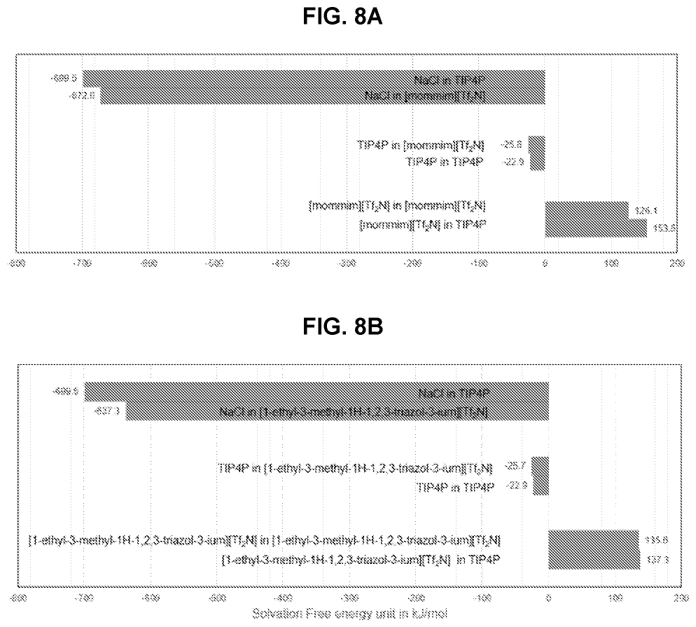

The beginning state of the calculation was the vacuum state, and the ending state of the calculation was the solution state. The vacuum state is widely used as the common reference for comparing the thermodynamic stability of the solute molecule in different status. In the simulation, all energy calculations are performed at 350K. Solvation free energies provide a method of determining thermodynamics stability in the respective environments for respective molecule, which effectively helps us understand the variable solvation tendencies. The calculated solvation free energies are listed in FIG. 8. The solvation free energy of NaCl in [mommim][Tf₂N] was −672.0 KJ/mol, and the solvation free energy of NaCl in [1,2,3-emtriz][Tf₂N] is −637.3KJ/mol, meanwhile, the solvation free energy of NaCl in water was-699.5KJ/mol. These values indicate that the NaCl will dissolve in the water phase and these two ILs will reject the NaCl from going into the IL phase. Similarly, the solvation free energy of water in water is −22.9KJ/mol, which is higher than the solvation free energy of values of −25.8 KJ/mol and −25.7KJ/mol, the values of water in [mommim][Tf₂N] and [1,2,3-emtriz][Tf₂N] respectively, and by comparing these values, it was concluded that the water will dissolve in these two ILs. To qualify as a suitable alternative solvent for DSE, the DS should not dissolve in water, and the solvation free energy comparison of IL in IL and IL in water supports this property. The solvation free energy of [mommim][Tf₂N] in [mommim][Tf₂N] is 126.1 KJ/mol and [mommim][Tf₂N] in water is 153.5 KJ/mol, while the [1-ethy-3-methyl-¹H-1,2,3-triazol-3-ium][Tf₂N] in [1,2,3-emtriz][Tf₂N] solvation free energy is 135.6 KJ/mol and [1,2,3-emtriz][Tf₂N] in water is 137.3 KJ/mol. Overall, these calculations are consistent with the experimental observations, illustrating that [mommim][Tf₂N] and [1,2,3-emtriz][Tf₂N] show satisfactory DS thermodynamics properties of these following facts: (1) water can dissolve in IL; (2) IL cannot dissolve in IL; (3) Na⁺ and Cl⁻ will only dissolve in water and will not dissolve in IL.

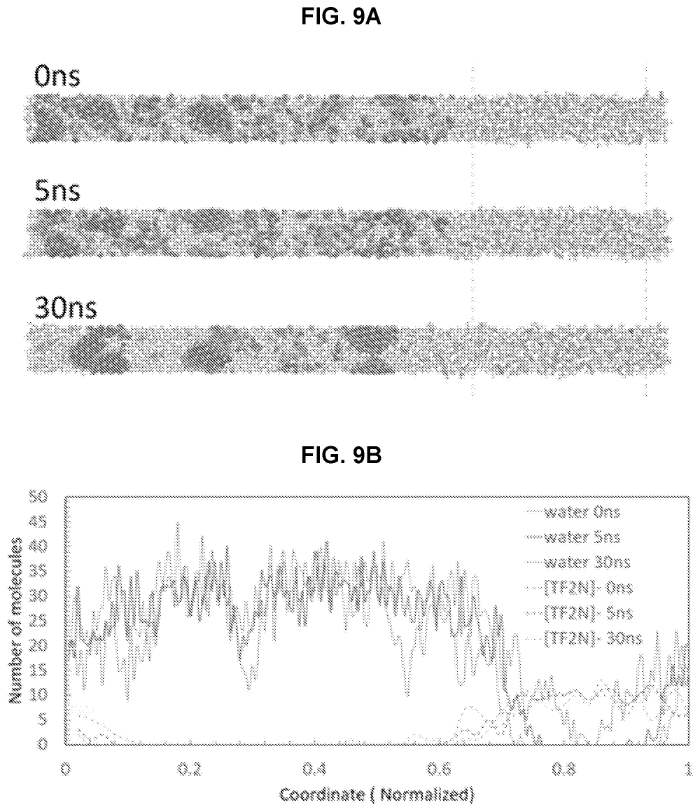

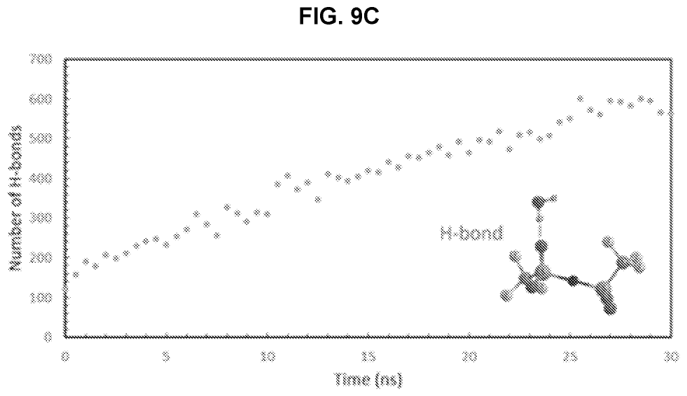

Besides the calculation of solvation free energy, ternary simulations with 25.8% NaCl and 3.7% NaCl water solutions in contact with [mommim][Tf₂N] and [1,2,3-emtriz][Tf₂N] were also performed. The simulations were run at 350K for a duration of 30 ns. FIG. 9A shows the snapshots of the ternary system, 25.8% NaCl water solution and [mommim][Tf₂N]. Throughout the simulation, almost all Na⁺ and Cl⁻ remain in the water phase, and only a small number of them diffused into the IL phase. Even the small number of Na⁺ and Cl⁻ ions that diffused into the IL phase are surrounded by water molecules. This scene can also explain the intrusion of droplets into the IL phase and the residual of Nat in the IL. A large number of water molecules diffused into the IL phase, but the number of IL that diffused into the water phase is only very limited. FIG. 9B shows the density profiles of water and IL at different times corresponding to FIG. 9A. Water diffusion into IL appeared much more prevalent than IL diffusion into water. This phenomenon coincides with the calculated solvation free energy value and the experiment results. The bonding nature between water and IL molecules was analyzed, and hydrogen bonds (FIG. 9C) were between water and the [Tf₂N]⁻ anions. Furthermore, as the amount of dissolved water increased, the number of hydrogen bonds also increased. In general, this phenomenon agrees with the calculation of the solvation free energy and the phenomenon observed in the experiment.

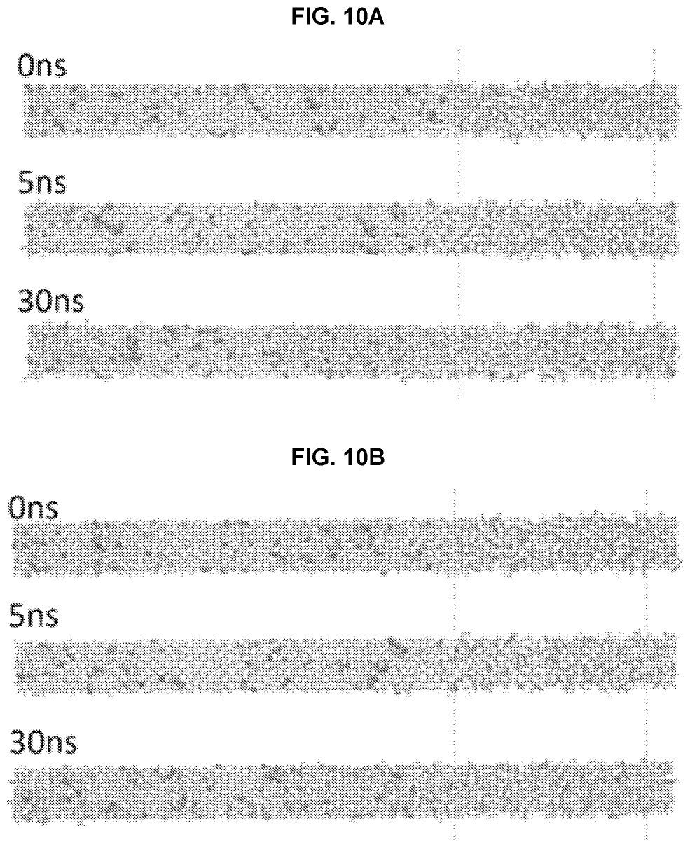

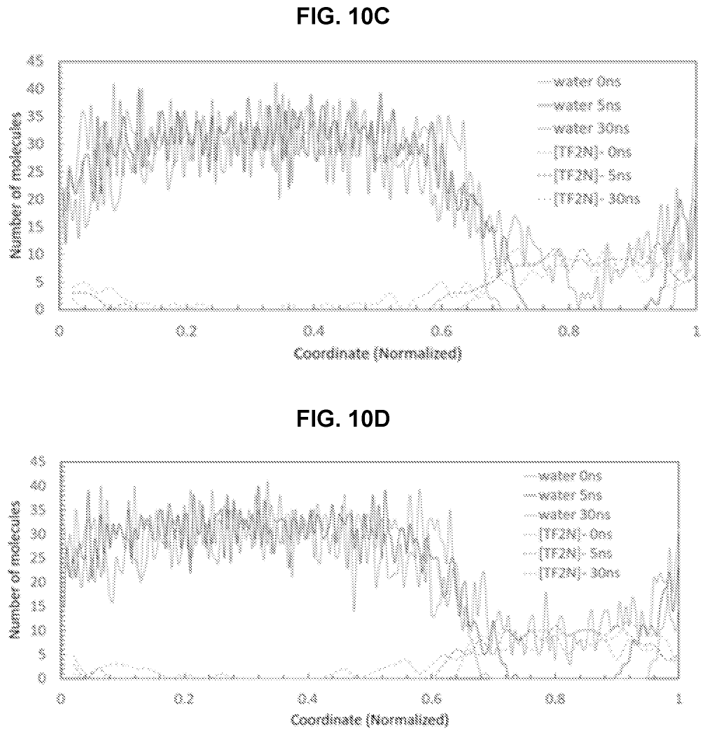

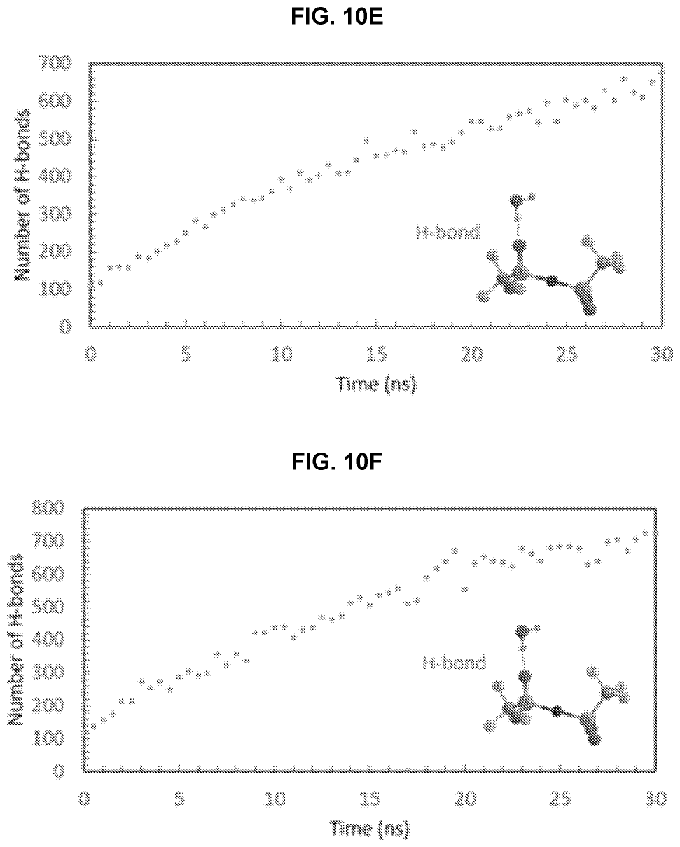

For the ternary simulation with 3.7% NaCl, a similar phenomenon was observed. FIG. 10A shows the snapshots of the simulation of the water, NaCl, and [mommim][Tf₂N] ternary system, and FIG. 10B shows the snapshots of the water, NaCl, and [1,2,3-emtriz][Tf₂N] system. FIG. 10C shows the density distribution of the simulation case shown in FIG. 10A. FIG. 10C shows the density distribution of the simulation case shown in FIG. 10D. FIG. 10E shows in the water, NaCl, and [mommim][Tf₂N] ternary system, the number of hydrogen bonds, and time relationship. FIG. 10F shows the relationship between the water, NaCl, and [1,2,3-emtriz][Tf₂N] ternary system, the number of hydrogen bonds, and time. In the two cases of the two ionic liquids in the saline having a NaCl concentration close to that of seawater, the simulation result also corresponds with the experiment result and calculated free energy. The simulation results in different concentrations shows that the ILs [mommim][Tf₂N] and [1,2,3-emtriz][Tf₂N] are capable of desalination of hypersaline and saline with more typical NaCl concentrations.

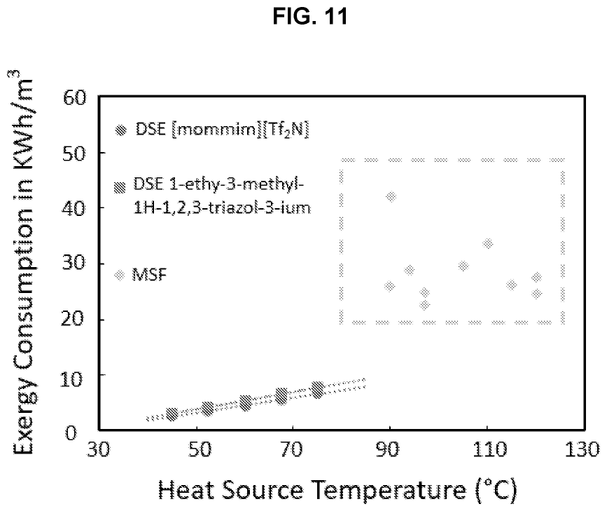

The DSE process can be operated in relatively mild temperatures, which enables the significant advantage of recycling the waste heat. From a heat source with Carnot efficiency, exergy is the maximum amount of work that can be extracted, so exergy is a valuable measure of the overall system efficiency when comparing the energy consumption of different desalination systems. As shown in FIG. 11, exergy consumption with [mommim][Tf₂N] and [1,2,3-emtriz][Tf₂N] increases with temperature. For example, the exergy cost of the DSE cycle with [mommim][Tf₂N] is 2.66kWh/m³ at 45° C. and 6.64 kWh/m³ at 75° C. The pumping power requirement for [mommim][Tf₂N] is 0.38kWh/m³ and for [1,2.3-emtriz][Tf₂N] this value is 0.45 kWh/m³. By comparing to the exergy cost of MSF technology, Elimelech & Phillip, Science 333:712-717 (2011) report that RO plants equipped with new techniques can reduce consumption as low as 2 kWh/m³. It is also reported a cost of 2.54 kWh/m³ is realized in the RO process. If operated at low temperatures, DSE has similar exergy costs as RO. Moreover, the DSE is more flexible and easier to miniaturize than an RO device because the energy cost for small-scale RO is high, which is caused by the difficulty of mechanical energy recovery.

The IL [mommim][Tf₂N] differs from [emim][Tf₂N] in that the ethyl side chain adorned on the imidazolium cation is replaced by the methoxymethyl side chain. This replacement of the ethyl group with the methoxymethyl side chain increases the hydrophilicity of the cation, thus increasing the IL's the solubility of water. However, the parameter that is most important is the water yield. The insertion of an oxygen atom in the side chain reduces the sensitivity of temperature to the solubility of water in the ionic liquid. The decreased salt ion rejection rate may also be attributed to the lengthened side chain, which caused a weaker interaction between the cation and the [Tf₂N]⁻ anion. Therefore, a stronger interaction may be achieved between [mommim][Cl] and [Na][Tf₂N] instead of rejecting the ion. As a result, the water yield and salt rejection rate are lower than [emim][Tf₂N] according to the experiment. For the case of [1,2,3-emtriz][Tf₂N], the change of the imidazolium pentagon cycle affects the solubility and temperature relationship, which caused a slender water yield drop. The replacement of the N atom with the C atom in the pentagon affects the charge distribution and caused a tighter interaction between the cation and Cl⁻, which leads to a small decrease in the rejection compared to [emim][Tf₂N]. In summary, the results demonstrate that ILs [mommim][Tf₂N] and [1.2,3-emtriz][Tf₂N] can efficiently remove salt ions from water, especially hypersaline.

### Example 3

### Task-Specific Ionic Liquid Directional Solvent Extraction Desalination of Di- and Poly-valent Salt Solutions

An ionic liquid (2 mL) and a 3.7-5.0 wt % aqueous solution of a divalent or polyvalent (e.g., metal) salt (2 mL) are mixed thoroughly at an elevated temperature (e.g., 45, 60, or 75° C.). The mixture is maintained at that temperature for ˜10 minutes until phase separation occurs. The IL-rich phase is collected, cooled to room temperature by removal of the heating element, and allowed to stand until phase separation occurs. The resulting biphasic mixture is separated by centrifugation (3000 rpm). The recovered water is used to calculate the fresh_{water} yield and analyzed for residual metal salt ions and ionic liquid. The divalent or polyvalent salt ion concentration is measured using a Perkin Elmer Optima 8000 ICP-OES, and residual IL content measured using a Waters TQD triple mass spectrometer coupled to an Acquity ultrahigh pressure liquid chromatography system.

What is claimed:

1. A method for desalinating water using a directional solvent extraction method (DSE), the method comprising:
forming a mixture by combining a salt solution comprising one or more ionic salts and water with an ionic liquid, the ionic liquid comprising:
an organic cation, wherein the organic cation is a phosphonium or an N-heterocyclic cation, and
an anion, wherein the anion is a sulfonyl imide, a sulfonamide, a sulfonate, a carboxylate, an alkoxide, a hydroxide, a borate, a phosphate, a halide, or an N-heterocyclic anion;
heating the mixture at a temperature of 40° C. to 90° C. to produce brine and an ionic solution, the ionic solution comprising water and the ionic liquid;
removing the brine from the ionic solution;
cooling the ionic solution to room temperature to produce a desalinated water phase and an ionic liquid phase; and
removing the desalinated water phase.

2. The method of claim 1, wherein the ionic salt comprises Nat, K⁺, NH⁴⁺, Ag⁺, Mg²⁺, Ca²⁺, Sr²⁺, Cd²⁺, Be²⁺, Ba²⁺, Pb²⁺, Hg²⁺, Zn²⁺, Cu²⁺, Ni²⁺, Cr³⁺, As³⁺, Tl³⁺, Al³⁺, Fe²⁺, Fe³⁺, Mn²⁺, Mn⁴⁺, Sb³⁺, Sb⁵⁺, or a combination thereof.

3. The method of claim 1, wherein the ionic salt comprises Cl⁻, Br⁻, I⁻, F⁻, NO₃⁻, NO₂⁻, CN⁻, PO₄³⁻, HPO₄²⁻, H₂PO₄⁻, PO₃³⁻, HPO₃²⁻, H₂PO₃₋, SO₄²⁻, HSO₄⁻, CO₃²⁻, HCO₃⁻, SiO₃²⁻, HSiO₃⁻, HBO₃²⁻, or H₂BO₃⁻, or a combination thereof.

4. The method of claim 1, wherein the ionic salt comprises NaCl, KCl, MgCl₂, CaCl₂, NaH₂PO₄, Na₂HPO₄, Na₃PO₄, NaHSO₄, Na₂SO₄, NaH₂PO₄, Na₂HPO₄, Na₃PO₄, NaHSO₄, Na₂SO₄, or a combination thereof.

5. The method of claim 1, wherein the salt solution comprises 3-15% by mass (wt %) of the ionic salt.

6. The method of claim 1, wherein heating the mixture occurs at a temperature of 45° C. to 75° C.

7. The method of claim 1, wherein the method further comprises recycling the ionic liquid phase.

8. The method of claim 1, wherein the organic cation is a phosphonium cation.

9. The method of claim 8, wherein the phosphonium cation is a tetraalkylphosphonium cation.

10. The method of claim 9, wherein the tetraalkylphosphonium cation is a tetralkyl phosphonium of formula: 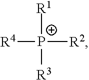
wherein R¹, R², R³, and R⁴ are each independently C₁₋₂₀alkyl.

11. The method of claim 10, wherein, R¹, R², and R³ are each independently C₄ salkyl and R⁴is C₄₋₁₄alkyl.

12. The method of claim 11, wherein R¹, R², R³, and R⁴ are each C₄alkyl.

13. The method of claim 11, wherein R¹, R², and R³ are each C₄alkyl and R⁴ is Csalkyl.

14. The method of claim 11, wherein R¹, R², and R³ are each Cealkyl and R⁴ is C₁₄alkyl.

15. The method of claim 1, wherein the organic cation is an N-heterocyclic cation.

16. The method of claim 15, wherein the N-heterocyclic cation is an imidazolium cation of formula: 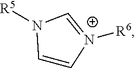
wherein R⁵ is C₁₋₄alkyl and R⁸ is C₁₋₁₀alkyl or —C₁₋₃alkylene-OC₁₋₄alkyl.

17. The method of claim 16, wherein R⁵ is methyl and R⁶ is C₁₋₅alkyl or —CH₂-OC₁₄alkyl.

18. The method of claim 17, wherein Re is C₂₋₄alkyl or —CH₂—OC₁₋₂alkyl.

19. The method of claim 15, wherein the N-heterocyclic cation is a triazolium cation of formula: 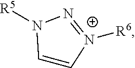
wherein R⁵ is C₁₄alkyl and R⁶ is C₁₋₁₀alkyl or —C₁₋₃alkylene-OC₁₋₄alkyl.

20. The method of claim 19, wherein R⁵ is methyl and R⁶ is C₂₋₄alkyl.

21. The method of claim 1, wherein the anion is bis(trifluoromethylsulfonyl)imide ([Tf2N]⁻) or tosylate ([OTs]⁻).

22. The method of claim 21, wherein the anion is [Tf2N]⁻.

23. The method of claim 21, wherein the anion is [OTs]⁻.

24. The method of claim 1, wherein the ionic liquid is:

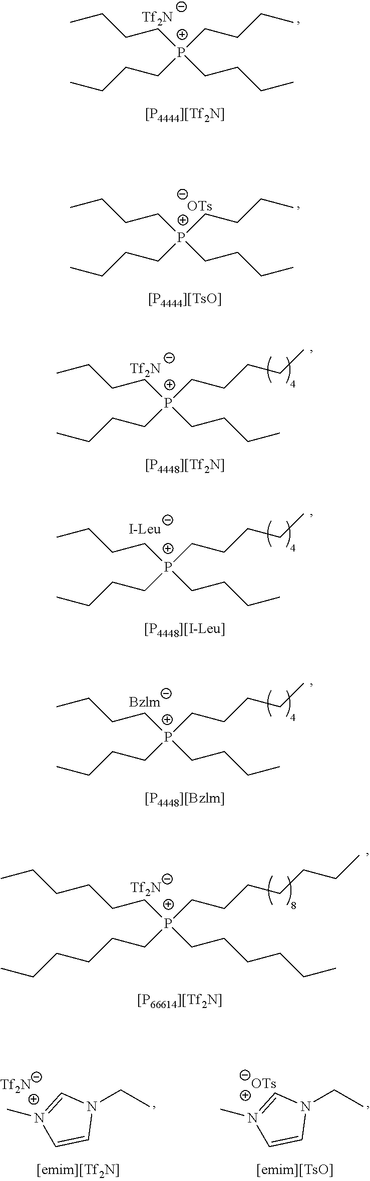

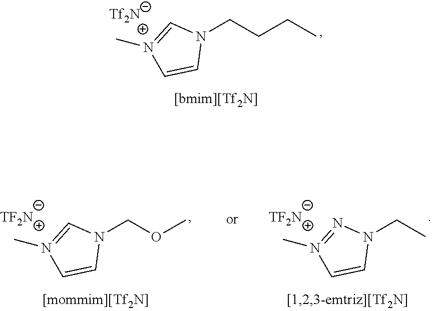

25. The method of claim 1, wherein the ionic liquid has an ion rejection rate of at least 70%.

26. The method of claim 1, wherein the ionic liquid has an ion rejection rate of at least 80%.

27. The method of claim 1, wherein the ionic liquid has an ion rejection rate of at least 90%.

28. Freshwater produced by the method of claim 1.

29. Use of the method of claim 1 to produce freshwater.

30. A method for desalinating seawater or groundwater with dissolved ionic salts, the method comprising:
forming a mixture by combining seawater or groundwater comprising dissolved ionic salts with an ionic liquid, wherein the ionic liquid is: 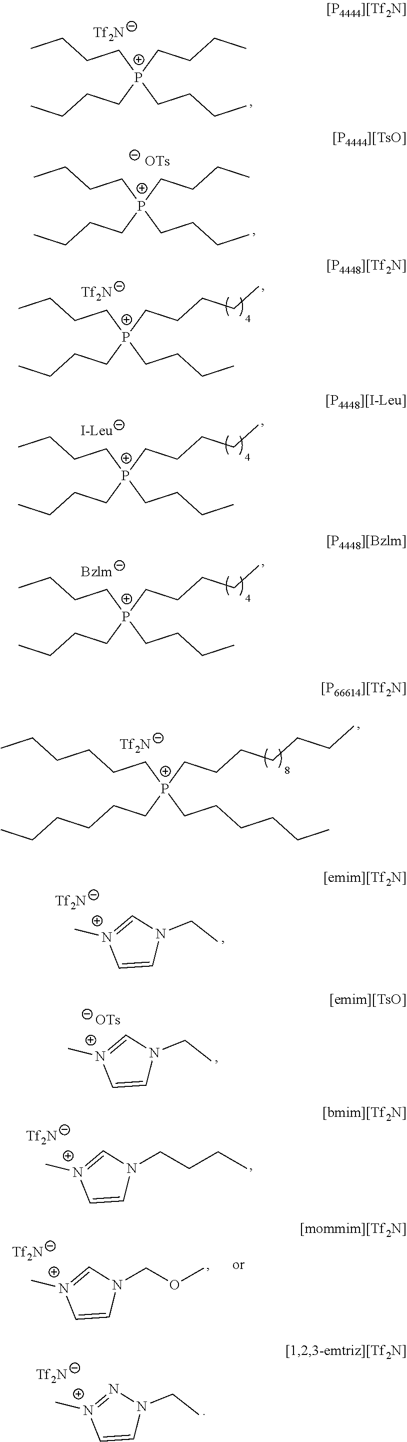
heating the mixture at a temperature of 40° C. to 90° C. to produce brine and an ionic solution, the ionic solution comprising water and the ionic liquid;
removing the brine from the ionic solution;
cooling the ionic solution to room temperature to produce a desalinated water phase and an ionic liquid phase; and
removing the desalinated water phase.
